SEQUENCE LISTING (4 sequences)

SEQ ID NO: 1 - protein, 595 aa, Homo sapiens
MPACCSCSDVFQYETNKVTRIQSMNYGTIKWFFHVIIFSYVCFALVSDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVYEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQLVVYIGSTLSYFGLAAVFIDFLIDTYSSNCCRSHIYPWCKCCQPCVVNEYYYRKKCESIVEPKPTLKYVSFVDESHIRMVNQQLLGRSLQDVKGQEVPRPAMDFTDLSRLPLALHDTPPIPGQPEEIQLLRKEATPRSRDSPVWCQCGSCLPSQLPESHRCLEELCCRKKPGACITTSELFRKLVLSRHVLQFLLLYQEPLLALDVDSTNSRLRHCAYRCYATWRFGSQDMADFAILPSCCRWRIRKEFPKSEGQYSGFKSPY

SEQ ID NO: 2 - protein, 290 aa, Homo sapiens
SDKLYQRKEPVISSVHTKVKGIAEVKEEIVENGVKKLVHSVFDTADYTFPLQGNSFFVMTNFLKTEGQEQRLCPEYPTRRTLCSSDRGCKKGWMDPQSKGIQTGRCVVYEGNQKTCEVSAWCPIEAVEEAPRPALLNSAENFTVLIKNNIDFPGHNYTTRNILPGLNITCTFHKTQNPQCPIFRLGDIFRETGDNFSDVAIQGGIMGIEIYWDCNLDRWFHHCRPKYSFRRLDDKTTNVSLYPGYNFRYAKYYKENNVEKRTLIKVFGIRFDILVFGTGGKFDIIQLVVY

SEQ ID NO: 3 - DNA, 3680 bp, Homo sapiens
  source 1..3680: mol_type = genomic DNA
gtcattggaggagcttgaagttaaagactcctgctaaaaaccagtacgtttcattttgcagttactgggagggggcttgctgtggccctgtcaggaagagtagagctctggtccagctccgcgcagggagggaggctgtcaccatgccggcctgctgcagctgcagtgatgttttccagtatgagacgaacaaagtcactcggatccagagcatgaattatggcaccattaagtggttcttccacgtgatcatcttttcctacgtttgctttgctctggtgagtgacaagctgtaccagcggaaagagcctgtcatcagttctgtgcacaccaaggtgaaggggatagcagaggtgaaagaggagatcgtggagaatggagtgaagaagttggtgcacagtgtctttgacaccgcagactacaccttccctttgcaggggaactctttcttcgtgatgacaaactttctcaaaacagaaggccaagagcagcggttgtgtcccgagtatcccacccgcaggacgctctgttcctctgaccgaggttgtaaaaagggatggatggacccgcagagcaaaggaattcagaccggaaggtgtgtagtgtatgaagggaaccagaagacctgtgaagtctctgcctggtgccccatcgaggcagtggaagaggccccccggcctgctctcttgaacagtgccgaaaacttcactgtgctcatcaagaacaatatcgacttccccggccacaactacaccacgagaaacatcctgccaggtttaaacatcacttgtaccttccacaagactcagaatccacagtgtcccattttccgactaggagacatcttccgagaaacaggcgataatttttcagatgtggcaattcagggcggaataatgggcattgagatctactgggactgcaacctagaccgttggttccatcactgccgtcccaaatacagtttccgtcgccttgacgacaagaccaccaacgtgtccttgtaccctggctacaacttcagatacgccaagtactacaaggaaaacaatgttgagaaacggactctgataaaagtcttcgggatccgttttgacatcctggtttttggcaccggaggaaaatttgacattatccagctggttgtgtacatcggctcaaccctctcctacttcggtctggccgctgtgttcatcgacttcctcatcgacacttactccagtaactgctgtcgctcccatatttatccctggtgcaagtgctgtcagccctgtgtggtcaacgaatactactacaggaagaagtgcgagtccattgtggagccaaagccgacattaaagtatgtgtcctttgtggatgaatcccacattaggatggtgaaccagcagctactagggagaagtctgcaagatgtcaagggccaagaagtcccaagacctgcgatggacttcacagatttgtccaggctgcccctggccctccatgacacacccccgattcctggacaaccagaggagatacagctgcttagaaaggaggcgactcctagatccagggatagccccgtctggtgccagtgtggaagctgcctcccatctcaactccctgagagccacaggtgcctggaggagctgtgctgccggaaaaagccgggggcctgcatcaccacctcagagctgttcaggaagctggtcctgtccagacacgtcctgcagttcctcctgctctaccaggagcccttgctggcgctggatgtggattccaccaacagccggctgcggcactgtgcctacaggtgctacgccacctggcgcttcggctcccaggacatggctgactttgccatcctgcccagctgctgccgctggaggatccggaaagagtttccgaagagtgaagggcagtacagtggcttcaagagtccttactgaagccaggcaccgtggctcacgtctgtaatcccagcgctttgggaggccgaggcaggcagatcacctgaggtcgggagttggagacccgcctggctaacaaggcgaaatcctgtctgtactaaaaatacaaaaatcagccagacatggtggcatgcacctgcaatcccagctactcgggaggctgaggcacaagaatcacttgaacccgggaggcagaggttgtagtgagcccagattgtgccactgctctccagcctgggaggcacagcaaactgtcccccaaaaaaaaaaaagagtccttaccaatagcaggggctgcagtagccatgttaacatgacatttaccagcaacttgaacttcacctgcaaagctctgtggccacattttcagccaaagggaaatatgctttcatcttctgttgctctctgtgtctgagagcaaagtgacctggttaaacaaaccagaatccctctacatggactcagagaaaagagattgagatgtaagtctcaactctgtccccaggaagttgtgtgaccctaggcctctcacctctgtgcctctgtctccttgttgcccaactactatctcagagatattgtgaggacaaattgagacagtgcacatgaactgtcttttaatgtgtaaagatctacatgaatgcaaaacatttcattatgaggtcagactaggataatgtccaactaaaaacaaacccttttcatcctggctggagaatgtggagaactaaaggtggccacaaattctttgacactcaagtcccccaagacctaagggttttatctcctccccttgaatatgggtggctctgattgctttatccaaaagtggaagtgacattgtgtcagtttcagatcctgatcttaagaggctgacagcttctacttgctgtcccttggaactcttgctatcggggaagccagacgccatttaaaagtctgcctatcctggccaggtgtggtggctcacacctgtaatcccagcactttgggagaccaaggcgggcggatcacttaaagtcaggagtccaagaccagactcgccaacatggtgaaaccgtatctctaataaaaatacaaaaattagctgggcatggtgcgggcacctgtagtcctagctatcaagaggctgagacaggagaaacacttgaacctgggaggtggaggttgcattgagctgagatcgtgccactgcactccaggctgggtgacagagcgagactccatctcaaaaaaaaaaaaaagaaaaaaaaaatgtctgcctatcctgagactgccctgctgtgaggaagcccaagcagtcacgtggacagtgcctgaccagccccagctttcaagccatccaagcccagtcaccaaacatgagagagaagaagccttcaggtgattctggactccactaacatatgactgataccgcatgatacatcccaagtgagaactgccccataaatccagaaaaccacattgctatcttaagtccctaagtttggggcttatttgttccacagcaacaggtaactggaacagagggcaagcctgatgaatgggcacacagactcagcccataccttccctggttctaatgttctcagggagcccggaccaaccctgggagcctcaggaacttaggtttccactggacagttctagaagggctatagaccaaatcaggtaactcaccagaccagccttggaatctatcaaatctaactgctgagctaccca

SEQ ID NO: 4 - protein, 595 aa, Homo sapiens; identical to SEQ ID NO: 1

(19) United States
(12) Patent Application Publication
(10) Pub. No.: US 2025/0129093 A1
(43) Pub. Date: Apr. 24, 2025

(54) COMPOSITIONS AND METHODS RELATING TO ATP/P2X7R SIGNALING INHIBITION

(71) Applicant: THE CHILDREN'S MEDICAL CENTER CORPORATION, Boston, MA (US)

(72) Inventors: Paolo FIORINA, Boston, MA (US); Moufida Ben NASR, Boston, MA (US)

(73) Assignee: THE CHILDREN'S MEDICAL CENTER CORPORATION, Boston, MA (US)

(21) Appl. No.: 18/835,427

(22) PCT Filed: Feb. 13, 2023
(86) PCT No.: PCT/US2023/012877

§ 371 Date: Aug. 2, 2024

Related U.S. Application Data
(60) Provisional application No. 63/310,312, filed on Feb. 15, 2022.

(51) Int. Cl. C07D 498/04; A61K 31/138; A61K 31/166; A61K 31/4015; A61K 31/4184; A61K 31/4985; A61K 31/505; A61K 31/53; A61K 31/7076; A61K 38/17; A61P 37/06; C07C 217/64; C07D 235/18; C07D 239/48

(52) U.S. Cl. CPC C07D 498/04; A61K 31/138; A61K 31/166; A61K 31/4015; A61K 31/4184; A61K 31/4985; A61K 31/505; A61K 31/53; A61K 31/7076; A61K 38/177; A61P 37/06; C07C 217/64; C07D 235/18; C07D 239/48

(57) ABSTRACT

Described herein are compositions, combinations, and methods relating to treating or preventing immune-related diseases, type 1 diabetes, transplant rejection, or lung fibrosis, or slowing or delaying aging processes or extending lifespan in a subject, including subjects with rs3751143.

### CROSS-REFERENCE TO RELATED APPLICATIONS

This application claims benefit under 35 U.S.C. § 119 (e) of U.S. Provisional Application No. 63/310,312 filed Feb. 15, 2022, the contents of which are incorporated herein by reference in their entirety.

### TECHNICAL FIELD

The technology described herein relates to inhibition of ATP/P2X7R and therapeutic applications thereof.

### BACKGROUND

Purinergic receptor-7 (P2X7R), primarily expressed on lymphocytes, senses adenosine 5′-triphosphate (ATP). ATP can be present in high concentration within cells and can also be released into the extracellular compartment as extracellular ATP (eATP) during cellular damage. The activation of eATP/P2X7R-axis is a fundamental step in immune cells activation and has been linked to T-cell activation and regulation, favoring T helper 1 (Th1) and Th17 generation and differentiation, antigen recognition and allograft rejection. eATP/P2X7R signaling has also been demonstrated to be involved in CD8+ T cell-mediated autoimmune responses in vitro and to facilitate the onset of autoimmune type 1 diabetes (TID) in NOD mice.

Accordingly, there is a need for inhibitors of eATP/P2X7R signaling in order to treat or prevent immune-mediated diseases such as type 1 diabetes, transplant rejection, lung fibrosis and aging.

### SUMMARY

Described herein are novel inhibitors of eATP/P2X7R signaling and methods of using them to treat or prevent immune-mediated diseases.

In one aspect of any of the embodiments, described herein is a composition comprising one or more of:

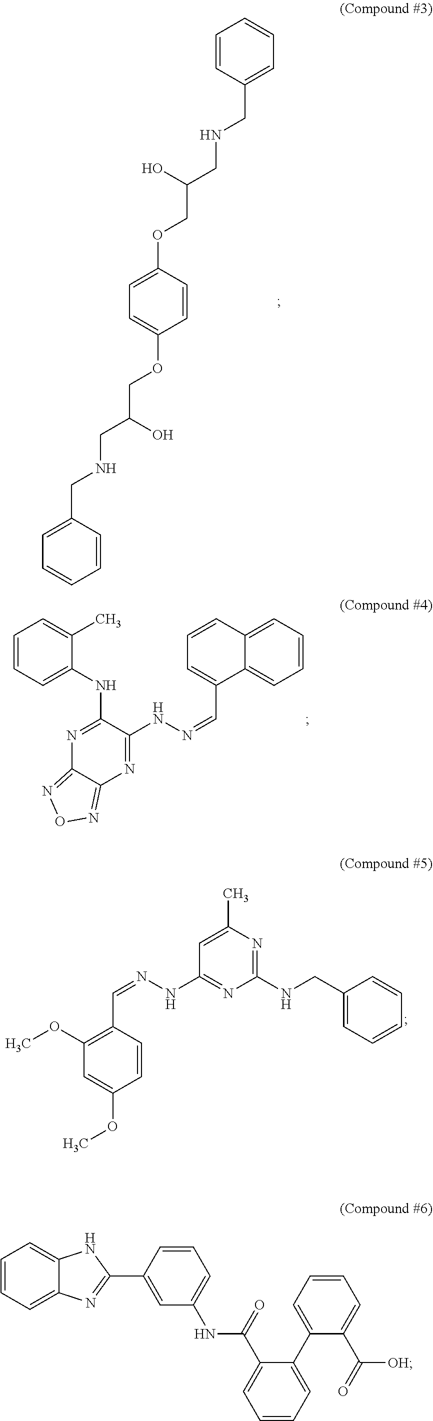

or a prodrug, isomer, enantiomer, diastereomer, racemate, tautomer, derivative, or salt thereof.

In one aspect of any of the embodiments, described herein is a composition or combination comprising:
a) the composition of any one of the preceding claims; and
b) one or more of:
   a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF).

In one aspect of any of the embodiments, described herein is a method of treating or preventing immune-related diseases, type 1 diabetes, transplant rejection, or lung fibrosis in a subject in need thereof, the method comprising administering a composition or combination described herein to the subject. In one aspect of any of the embodiments, described herein is a method of slowing or delaying aging processes or extending lifespan in a subject, the method comprising administering a composition or combination described herein to the subject.

In one aspect of any of the embodiments, described herein is a composition or combination described herein for use in a method of treating or preventing immune-related diseases, type 1 diabetes, transplant rejection, or lung fibrosis in a subject in need thereof. In one aspect of any of the embodiments, described herein is a composition or combination as described herein for use in a method of slowing or delaying aging processes or extending lifespan in a subject.

In some embodiments of any of the aspects, the subject has or has been determined to have rs3751143. In some embodiments of any of the aspects, the subject has hyper-Th17 syndrome.

In one aspect of any of the embodiments, described herein is a method of treating or preventing immune-related diseases, type 1 diabetes, transplant rejection, or lung fibrosis in a subject in need thereof, the method comprising administering a composition comprising one or more of oATP, CE-224535, AZD9056, GSK1482160, a polypeptide comprising the ectodomain of P2X7R, and a P2X7R soluble protein to the subject; wherein the subject has or has been determined to have rs3751143. In one aspect of any of the embodiments, described herein is a composition comprising one or more of oATP, CE-224535, AZD9056, GSK1482160, a polypeptide comprising the ectodomain of P2X7R, and a P2X7R soluble protein to the subject for use in a method of treating or preventing immune-related diseases, type 1 diabetes, transplant rejection, or lung fibrosis in a subject in need thereof; wherein the subject has or has been determined to have rs3751143.

In one aspect of any of the embodiments, described herein is a method of slowing or delaying aging processes or extending lifespan in a subject, the method comprising administering a composition comprising one or more of oATP, CE-224535, AZD9056, GSK1482160, a polypeptide comprising the ectodomain of P2X7R, and a P2X7R soluble protein to the subject; wherein the subject has or has been determined to have rs3751143. In one aspect of any of the embodiments, described herein is a composition comprising one or more of oATP, CE-224535, AZD9056, GSK1482160, a polypeptide comprising the ectodomain of P2X7R, and a P2X7R soluble protein to the subject for use in a method of slowing or delaying aging processes or extending lifespan in a subject; wherein the subject has or has been determined to have rs3751143.

In some embodiments of any of the aspects, the subject has hyper-Th17 syndrome. In some embodiments of any of the aspects, the treatment or prevention of type 1 diabetes comprises slowing the progression or onset of type 1 diabetes. In some embodiments of any of the aspects, the subject has previously received a transplant of an organ and/or cells and the treatment or prevention of transplant rejection comprises suppressing or delaying rejection of the transplant.

### BRIEF DESCRIPTION OF THE DRAWINGS

FIG. 1 depicts bioinformatic screening of small molecules capable of selectively binding eATP, e.g, ATP binding proteins/enzymes that are <200res.

FIG. 2 demonstrates the effects of new small molecules that target eATP/P2X7R on PBMCs. Compound #5 demonstrates an effect comparable to that of human P2X7R inhibitor CE-224,535 and P2X7 receptor antagonist oATP on the expression of Th2-related factors GATA3, IL-4 and IRF4.

FIGS. 3A-3B depict graphs of responses to P2X7R signaling inhibitors. FIG. 3A demonstrates that treatment with a P2X7R inhibitor (oATP) prolongs allograft survival in P2X7R−/− mice receiving a Bm12 heart. FIG. 3B demonstrates that Th17 generation is partially abrogated in vitro in CD4+ T cells obtained from individuals carrying the P2X7R mutation by using a P2X7R inhibitor (oATP or CE 224,535).

FIG. 4 demonstrates that increased survival is achieved by administering oATP in B6 mice as compared to untreated controls (p=0.01).

### DETAILED DESCRIPTION

Described herein are small molecules that can bind ATP, e.g., extracellular ATP, and thereby inhibit eATP/P2X7R signaling. The purine ATP is a small molecule present at high concentrations within cells and released after cell damage or death and immune cell activation; it acts as a danger signal and potent chemotactic mediator. ATP is abundant at inflammation sites and is sensed by ionotropic purinergic P2X receptors (seven receptors named P2X1-P2X7, or P2XRs). P2X receptors can function as calcium channels, and autocrine activation of these receptors can facilitate calcium influx and downstream signaling. In leukocytes, P2XRs can regulate cytokine production, activation, and apoptosis, thus constituting an “autocrine alerting system”. In particular, P2X7R can serve as a signal amplification mechanism for antigen recognition. As used herein, “ATP/P2X7R signaling” or “eATP/P2X7R signaling” refers to the specific binding of the cell surface receptor P2X7R to ATP (e.g., extracellular ATP) and the downstream events triggered by that recognition, e.g., phosphorylation of STAT3, Th1 and Th17 differentiation, and CD4+ proliferation and IFN-γ production.

As used herein “P2X7R” refers to polypeptide that forms a homomeric trimeric P2X7 receptor, which is a ligand-gated cation channel that opens in response to ATP binding and causes depolarization of the cell. P2X7R is also referred to as P2RX7 and P2X7. The sequences of P2X7R for a number of species are known in the art, e.g., human P2X7R (NCBI Gene ID No: 5027; mRNA (NCBI Ref Seq: NM_002562, SEQ ID NO: 3); polypeptide (NCBI Ref Seq: NP_002553, SEQ ID NO: 4).

P2X7R comprises an ectodomain (e.g., SEQ ID NO:2), a transmembrane domain, and an intracellular domain. These domains are known in the art and annotated in, e.g., the NCBI database as of filing of this application. A protein comprising at least one ectodomain of P2X7R and not comprising the transmembrane domain of P2X7R will be soluble. A “soluble P2X7R” protein excludes the transmembrane domain of P2X7R. Such a soluble P2X7R protein, or an exogenous or ectopic polypeptide comprising the ectodomain of P2X7R will bind to extracellular ATP and thereby inhibit eATP/P2X7R signaling.

In some embodiments, a soluble P2X7R polypeptide can comprise a polypeptide having the sequence of SEQ ID NO: 2. In some embodiments, a soluble P2X7R polypeptide can comprise repeats of the ectodomain of P2X7R, e.g. two copies of the ectodomain of P2X7R, three copies of the ectodomain of P2X7R, or more copies of the ectodomain of P2X7R. In some embodiments, a soluble P2X7R polypeptide can be a fusion polypeptide.

| SEQ | | |
|---|---|---|
| ID | | |
| NO | | |
| 1 | Met Pro Ala Cys Cys Ser Cys Ser Asp Val Phe Gln Tyr Glu Thr Asn Lys Val Thr | |
| | Arg Ile Gln Ser Met Asn Tyr Gly Thr Ile Lys Trp Phe Phe His Val Ile Ile Phe Ser | |
| | Tyr Val Cys Phe Ala Leu Val Ser Asp Lys Leu Tyr Gln Arg Lys Glu Pro Val Ile | |
| | Ser Ser Val His Thr Lys Val Lys Gly Ile Ala Glu Val Lys Glu Glu Ile Val Glu Asn | |
| | Gly Val Lys Lys Leu Val His Ser Val Phe Asp Thr Ala Asp Tyr Thr Phe Pro Leu | |
| | Gln Gly Asn Ser Phe Phe Val Met Thr Asn Phe Leu Lys Thr Glu Gly Gln Glu Gln | |
| | Arg Leu Cys Pro Glu Tyr Pro Thr Arg Arg Thr Leu Cys Ser Ser Asp Arg Gly Cys | |
| | Lys Lys Gly Trp Met Asp Pro Gln Ser Lys Gly Ile Gln Thr Gly Arg Cys Val Val | |
| | Tyr Glu Gly Asn Gln Lys Thr Cys Glu Val Ser Ala Trp Cys Pro Ile Glu Ala Val | |
| | Glu Glu Ala Pro Arg Pro Ala Leu Leu Asn Ser Ala Glu Asn Phe Thr Val Leu Ile | |
| | Lys Asn Asn Ile Asp Phe Pro Gly His Asn Tyr Thr Thr Arg Asn Ile Leu Pro Gly | |
| | Leu Asn Ile Thr Cys Thr Phe His Lys Thr Gln Asn Pro Gln Cys Pro Ile Phe Arg | |
| | Leu Gly Asp Ile Phe Arg Glu Thr Gly Asp Asn Phe Ser Asp Val Ala Ile Gln Gly | |
| | Gly Ile Met Gly Ile Glu Ile Tyr Trp Asp Cys Asn Leu Asp Arg Trp Phe His His | |
| | Cys Arg Pro Lys Tyr Ser Phe Arg Arg Leu Asp Asp Lys Thr Thr Asn Val Ser Leu | |
| | Tyr Pro Gly Tyr Asn Phe Arg Tyr Ala Lys Tyr Tyr Lys Glu Asn Asn Val Glu Lys | |
| | Arg Thr Leu Ile Lys Val Phe Gly Ile Arg Phe Asp Ile Leu Val Phe Gly Thr Gly Gly | |
| | Lys Phe Asp Ile Ile Gln Leu Val Val Tyr Ile Gly Ser Thr Leu Ser Tyr Phe Gly Leu | |
| | Ala Ala Val Phe Ile Asp Phe Leu Ile Asp Thr Tyr Ser Ser Asn Cys Cys Arg Ser His | |
| | Ile Tyr Pro Trp Cys Lys Cys Cys Gln Pro Cys Val Val Asn Glu Tyr Tyr Tyr Arg | |
| | Lys Lys Cys Glu Ser Ile Val Glu Pro Lys Pro Thr Leu Lys Tyr Val Ser Phe Val | |
| | Asp Glu Ser His Ile Arg Met Val Asn Gln Gln Leu Leu Gly Arg Ser Leu Gln Asp | |
| | Val Lys Gly Gln Glu Val Pro Arg Pro Ala Met Asp Phe Thr Asp Leu Ser Arg Leu | |
| | Pro Leu Ala Leu His Asp Thr Pro Pro Ile Pro Gly Gln Pro Glu Glu Ile Gln Leu Leu | |
| | Arg Lys Glu Ala Thr Pro Arg Ser Arg Asp Ser Pro Val Trp Cys Gln Cys Gly Ser | |
| | Cys Leu Pro Ser Gln Leu Pro Glu Ser His Arg Cys Leu Glu Glu Leu Cys Cys Arg | |
| | Lys Lys Pro Gly Ala Cys Ile Thr Thr Ser Glu Leu Phe Arg Lys Leu Val Leu Ser | |
| | Arg His Val Leu Gln Phe Leu Leu Leu Tyr Gln Glu Pro Leu Leu Ala Leu Asp Val | |
| | Asp Ser Thr Asn Ser Arg Leu Arg His Cys Ala Tyr Arg Cys Tyr Ala Thr Trp Arg | |
| | Phe Gly Ser Gln Asp Met Ala Asp Phe Ala Ile Leu Pro Ser Cys Cys Arg Trp Arg | |
| | Ile Arg Lys Glu Phe Pro Lys Ser Glu Gly Gln Tyr Ser Gly Phe Lys Ser Pro Tyr | |
| 2 | Ser Asp Lys Leu Tyr Gln Arg Lys Glu Pro Val Ile Ser Ser Val His Thr Lys Val Lys | |
| | Gly Ile Ala Glu Val Lys Glu Glu Ile Val Glu Asn Gly Val Lys Lys Leu Val His Ser | |
| | Val Phe Asp Thr Ala Asp Tyr Thr Phe Pro Leu Gln Gly Asn Ser Phe Phe Val Met | |
| | Thr Asn Phe Leu Lys Thr Glu Gly Gln Glu Gln Arg Leu Cys Pro Glu Tyr Pro Thr | |
| | Arg Arg Thr Leu Cys Ser Ser Asp Arg Gly Cys Lys Lys Gly Trp Met Asp Pro Gln | |
| | Ser Lys Gly Ile Gln Thr Gly Arg Cys Val Val Tyr Glu Gly Asn Gln Lys Thr Cys | |
| | Glu Val Ser Ala Trp Cys Pro Ile Glu Ala Val Glu Glu Ala Pro Arg Pro Ala Leu | |
| | Leu Asn Ser Ala Glu Asn Phe Thr Val Leu Ile Lys Asn Asn Ile Asp Phe Pro Gly | |
| | His Asn Tyr Thr Thr Arg Asn Ile Leu Pro Gly Leu Asn Ile Thr Cys Thr Phe His | |
| | Lys Thr Gln Asn Pro Gln Cys Pro Ile Phe Arg Leu Gly Asp Ile Phe Arg Glu Thr | |
| | Gly Asp Asn Phe Ser Asp Val Ala Ile Gln Gly Gly Ile Met Gly Ile Glu Ile Tyr Trp | |
| | Asp Cys Asn Leu Asp Arg Trp Phe His His Cys Arg Pro Lys Tyr Ser Phe Arg Arg | |
| | Leu Asp Asp Lys Thr Thr Asn Val Ser Leu Tyr Pro Gly Tyr Asn Phe Arg Tyr Ala | |
| | Lys Tyr Tyr Lys Glu Asn Asn Val Glu Lys Arg Thr Leu Ile Lys Val Phe Gly Ile | |
| | Arg Phe Asp Ile Leu Val Phe Gly Thr Gly Gly Lys Phe Asp Ile Ile Gln Leu Val Val | |
| | Tyr | |
| 3 | gtcattggag gagcttgaag ttaaagactc ctgctaaaaa ccagtacgtt tcattttgca | 60 |
| | gttactggga gggggcttgc tgtggccctg tcaggaagag tagagctctg gtccagctcc | 120 |
| | gcgcagggag ggaggctgtc accatgccgg cctgctgcag ctgcagtgat gttttccagt | 180 |
| | atgagacgaa caaagtcact cggatccaga gcatgaatta tggcaccatt aagtggttct | 240 |
| | tccacgtgat catcttttcc tacgtttgct ttgctctggt gagtgacaag ctgtaccagc | 300 |
| | ggaaagagcc tgtcatcagt tctgtgcaca ccaaggtgaa ggggatagca gaggtgaaag | 360 |
| | aggagatcgt ggagaatgga gtgaagaagt tggtgcacag tgtctttgac accgcagact | 420 |
| | acaccttccc tttgcagggg aactctttct tcgtgatgac aaactttctc aaaacagaag | 480 |
| | gccaagagca gcggttgtgt cccgagtatc ccacccgcag gacgctctgt tcctctgacc | 540 |
| | gaggttgtaa aaagggatgg atggacccgc agagcaaagg aattcagacc ggaaggtgtg | 600 |
| | tagtgtatga agggaaccag aagacctgtg aagtctctgc ctggtgcccc atcgaggcag | 660 |
| | tggaagaggc cccccggcct gctctcttga acagtgccga aaacttcact gtgctcatca | 720 |
| | agaacaatat cgacttcccc ggccacaact acaccacgag aaacatcctg ccaggtttaa | 780 |
| | acatcacttg taccttccac aagactcaga atccacagtg tcccattttc cgactaggag | 840 |
| | acatcttccg agaaacaggc gataattttt cagatgtggc aattcagggc ggaataatgg | 900 |
| | gcattgagat ctactgggac tgcaacctag accgttggtt ccatcactgc cgtcccaaat | 960 |
| | acagtttccg tcgccttgac gacaagacca ccaacgtgtc cttgtaccct ggctacaact | 1020 |
| | tcagatacgc caagtactac aaggaaaaca atgttgagaa acggactctg ataaaagtct | 1080 |
| | tcgggatccg ttttgacatc ctggtttttg gcaccggagg aaaatttgac attatccagc | 1140 |
| | tggttgtgta catcggctca accctctcct acttcggtct ggccgctgtg ttcatcgact | 1200 |
| | tcctcatcga cacttactcc agtaactgct gtcgctccca tatttatccc tggtgcaagt | 1260 |
| | gctgtcagcc ctgtgtggtc aacgaatact actacaggaa gaagtgcgag tccattgtgg | 1320 |
| | agccaaagcc gacattaaag tatgtgtcct ttgtggatga atcccacatt aggatggtga | 1380 |
| | accagcagct actagggaga agtctgcaag atgtcaaggg ccaagaagtc ccaagacctg | 1440 |
| | cgatggactt cacagatttg tccaggctgc ccctggccct ccatgacaca cccccgattc | 1500 |
| | ctggacaacc agaggagata cagctgctta gaaaggaggc gactcctaga tccagggata | 1560 |
| | gccccgtctg gtgccagtgt ggaagctgcc tcccatctca actccctgag agccacaggt | 1620 |
| | gcctggagga gctgtgctgc cggaaaaagc cgggggcctg catcaccacc tcagagctgt | 1680 |
| | tcaggaagct ggtcctgtcc agacacgtcc tgcagttcct cctgctctac caggagccct | 1740 |
| | tgctggcgct ggatgtggat tccaccaaca gccggctgcg gcactgtgcc tacaggtgct | 1800 |
| | acgccacctg gcgcttcggc tcccaggaca tggctgactt tgccatcctg cccagctgct | 1860 |
| | gccgctggag gatccggaaa gagtttccga agagtgaagg gcagtacagt ggcttcaaga | 1920 |
| | gtccttactg aagccaggca ccgtggctca cgtctgtaat cccagcgctt tgggaggccg | 1980 |
| | aggcaggcag atcacctgag gtcgggagtt ggagacccgc ctggctaaca aggcgaaatc | 2040 |
| | ctgtctgtac taaaaataca aaaatcagcc agacatggtg gcatgcacct gcaatcccag | 2100 |
| | ctactcggga ggctgaggca caagaatcac ttgaacccgg gaggcagagg ttgtagtgag | 2160 |
| | cccagattgt gccactgctc tccagcctgg gaggcacagc aaactgtccc ccaaaaaaaa | 2220 |
| | aaaagagtcc ttaccaatag caggggctgc agtagccatg ttaacatgac atttaccagc | 2280 |
| | aacttgaact tcacctgcaa agctctgtgg ccacattttc agccaaaggg aaatatgctt | 2340 |
| | tcatcttctg ttgctctctg tgtctgagag caaagtgacc tggttaaaca aaccagaatc | 2400 |
| | cctctacatg gactcagaga aaagagattg agatgtaagt ctcaactctg tccccaggaa | 2460 |
| | gttgtgtgac cctaggcctc tcacctctgt gcctctgtct ccttgttgcc caactactat | 2520 |
| | ctcagagata ttgtgaggac aaattgagac agtgcacatg aactgtcttt taatgtgtaa | 2580 |
| | agatctacat gaatgcaaaa catttcatta tgaggtcaga ctaggataat gtccaactaa | 2640 |
| | aaacaaaccc ttttcatcct ggctggagaa tgtggagaac taaaggtggc cacaaattct | 2700 |
| | ttgacactca agtcccccaa gacctaaggg ttttatctcc tccccttgaa tatgggtggc | 2760 |
| | tctgattgct ttatccaaaa gtggaagtga cattgtgtca gtttcagatc ctgatcttaa | 2820 |
| | gaggctgaca gcttctactt gctgtccctt ggaactcttg ctatcgggga agccagacgc | 2880 |
| | catttaaaag tctgcctatc ctggccaggt gtggtggctc acacctgtaa tcccagcact | 2940 |
| | ttgggagacc aaggcgggcg gatcacttaa agtcaggagt ccaagaccag actcgccaac | 3000 |
| | atggtgaaac cgtatctcta ataaaaatac aaaaattagc tgggcatggt gcgggcacct | 3060 |
| | gtagtcctag ctatcaagag gctgagacag gagaaacact tgaacctggg aggtggaggt | 3120 |
| | tgcattgagc tgagatcgtg ccactgcact ccaggctggg tgacagagcg agactccatc | 3180 |
| | tcaaaaaaaa aaaaaagaaa aaaaaaatgt ctgcctatcc tgagactgcc ctgctgtgag | 3240 |
| | gaagcccaag cagtcacgtg gacagtgcct gaccagcccc agctttcaag ccatccaagc | 3300 |
| | ccagtcacca aacatgagag agaagaagcc ttcaggtgat tctggactcc actaacatat | 3360 |
| | gactgatacc gcatgataca tcccaagtga gaactgcccc ataaatccag aaaaccacat | 3420 |
| | tgctatctta agtccctaag tttggggctt atttgttcca cagcaacagg taactggaac | 3480 |
| | agagggcaag cctgatgaat gggcacacag actcagccca taccttccct ggttctaatg | 3540 |
| | ttctcaggga gcccggacca accctgggag cctcaggaac ttaggtttcc actggacagt | 3600 |
| | tctagaaggg ctatagacca aatcaggtaa ctcaccagac cagccttgga atctatcaaa | 3660 |
| | tctaactgct gagctaccca | 3680 |

| | |
|---|---|
| 4 | Met Pro Ala Cys Cys Ser Cys Ser Asp Val Phe Gln Tyr Glu Thr Asn Lys Val Thr |
| | Arg Ile Gln Ser Met Asn Tyr Gly Thr Ile Lys Trp Phe Phe His Val Ile Ile Phe Ser |
| | Tyr Val Cys Phe Ala Leu Val Ser Asp Lys Leu Tyr Gln Arg Lys Glu Pro Val Ile |
| | Ser Ser Val His Thr Lys Val Lys Gly Ile Ala Glu Val Lys Glu Glu Ile Val Glu Asn |
| | Gly Val Lys Lys Leu Val His Ser Val Phe Asp Thr Ala Asp Tyr Thr Phe Pro Leu |
| | Gln Gly Asn Ser Phe Phe Val Met Thr Asn Phe Leu Lys Thr Glu Gly Gln Glu Gln |
| | Arg Leu Cys Pro Glu Tyr Pro Thr Arg Arg Thr Leu Cys Ser Ser Asp Arg Gly Cys |
| | Lys Lys Gly Trp Met Asp Pro Gln Ser Lys Gly Ile Gln Thr Gly Arg Cys Val Val |
| | Tyr Glu Gly Asn Gln Lys Thr Cys Glu Val Ser Ala Trp Cys Pro Ile Glu Ala Val |
| | Glu Glu Ala Pro Arg Pro Ala Leu Leu Asn Ser Ala Glu Asn Phe Thr Val Leu Ile |
| | Lys Asn Asn Ile Asp Phe Pro Gly His Asn Tyr Thr Thr Arg Asn Ile Leu Pro Gly |
| | Leu Asn Ile Thr Cys Thr Phe His Lys Thr Gln Asn Pro Gln Cys Pro Ile Phe Arg |
| | Leu Gly Asp Ile Phe Arg Glu Thr Gly Asp Asn Phe Ser Asp Val Ala Ile Gln Gly |
| | Gly Ile Met Gly Ile Glu Ile Tyr Trp Asp Cys Asn Leu Asp Arg Trp Phe His His |
| | Cys Arg Pro Lys Tyr Ser Phe Arg Arg Leu Asp Asp Lys Thr Thr Asn Val Ser Leu |
| | Tyr Pro Gly Tyr Asn Phe Arg Tyr Ala Lys Tyr Tyr Lys Glu Asn Asn Val Glu Lys |
| | Arg Thr Leu Ile Lys Val Phe Gly Ile Arg Phe Asp Ile Leu Val Phe Gly Thr Gly Gly |
| | Lys Phe Asp Ile Ile Gln Leu Val Val Tyr Ile Gly Ser Thr Leu Ser Tyr Phe Gly Leu |
| | Ala Ala Val Phe Ile Asp Phe Leu Ile Asp Thr Tyr Ser Ser Asn Cys Cys Arg Ser His |
| | Ile Tyr Pro Trp Cys Lys Cys Cys Gln Pro Cys Val Val Asn Glu Tyr Tyr Tyr Arg |
| | Lys Lys Cys Glu Ser Ile Val Glu Pro Lys Pro Thr Leu Lys Tyr Val Ser Phe Val |
| | Asp Glu Ser His Ile Arg Met Val Asn Gln Gln Leu Leu Gly Arg Ser Leu Gln Asp |
| | Val Lys Gly Gln Glu Val Pro Arg Pro Ala Met Asp Phe Thr Asp Leu Ser Arg Leu |
| | Pro Leu Ala Leu His Asp Thr Pro Pro Ile Pro Gly Gln Pro Glu Glu Ile Gln Leu Leu |
| | Arg Lys Glu Ala Thr Pro Arg Ser Arg Asp Ser Pro Val Trp Cys Gln Cys Gly Ser |
| | Cys Leu Pro Ser Gln Leu Pro Glu Ser His Arg Cys Leu Glu Glu Leu Cys Cys Arg |
| | Lys Lys Pro Gly Ala Cys Ile Thr Thr Ser Glu Leu Phe Arg Lys Leu Val Leu Ser |
| | Arg His Val Leu Gln Phe Leu Leu Leu Tyr Gln Glu Pro Leu Leu Ala Leu Asp Val |
| | Asp Ser Thr Asn Ser Arg Leu Arg His Cys Ala Tyr Arg Cys Tyr Ala Thr Trp Arg |
| | Phe Gly Ser Gln Asp Met Ala Asp Phe Ala Ile Leu Pro Ser Cys Cys Arg Trp Arg |
| | Ile Arg Lys Glu Phe Pro Lys Ser Glu Gly Gln Tyr Ser Gly Phe Lys Ser Pro Tyr |

As used herein, the term “fusion protein” or “fusion polypeptide” refers to a protein created by joining two genes or two proteins/peptides together. In a fusion protein, the two proteins can be joined together with a linker or spacer peptide added between the two proteins. In some embodiments, a soluble P2X7R polypeptide can be a fusion polypeptide further comprising an Ig protein, or Fc domain.

The compositions and methods described herein relate to inhibitors of eATP/P2X7R signaling. As used herein, the term “inhibitor” refers to an agent which can decrease the expression and/or activity of the target (e.g. eATP/P2X7R signaling), e.g. by at least 10% or more, e.g. by 10% or more, 50% or more, 70% or more, 80% or more, 90% or more, 95% or more, or 98% or more. The efficacy of an inhibitor of, for example, eATP/P2X7R signaling, e.g. its ability to decrease the level and/or activity of eATP/P2X7R signaling, can be determined, e.g. by measuring the level of phosphorylated STAT3, Th1 or Th17 differentiation, and/or CD4+ proliferation. Methods for measuring the level of, e.g. eATP/P2X7R signaling, a specific molecule (e.g. ATP), and/or a given mRNA and/or polypeptide are known to one of skill in the art, e.g. RTPCR can be used to determine the level of RNA and Western blotting with an antibody can be used to determine the level of a polypeptide.

In some embodiments, an inhibitor will directly bind to the targeted factor, e.g. to ATP or to P2X7R (or a nucleic acid encoding P2X7R). In some embodiments, an inhibitor will directly result in the cleavage of the targeted factor's mRNA, e.g., via RNA interference. In some embodiments, an inhibitor can act in a competitive manner to inhibit activity of the targeted factor. In some embodiments, an inhibitor can comprise a portion of the target factor and act as a competitive or dominant negative factor for interactions normally involving the targeted factor.

In some embodiments, an inhibitor of eATP/P2X7R signaling, e.g., an inhibitor of eATP or P2X7R can be an inhibitor that is exogenous to the subject and/or cells being treated, e.g., beta cells. In some embodiments, an inhibitor of eATP/P2X7R signaling, e.g., an inhibitor of eATP and/or P2X7R can be an inhibitor that is ectopic to the subject and/or cells being treated, e.g., beta cells. The term “exogenous” refers to a substance present in a cell other than its native source. A substance will be considered exogenous if it is introduced into a cell or an ancestor of the cell from which the cell has inherited the substance. In contrast, the term “endogenous” refers to a substance that is native to the biological system or cell (e.g. the beta cell and/or target cell). As used herein, “ectopic” refers to a substance that is found in an unusual location and/or amount. An ectopic substance can be one that is normally found in a given cell, but at a much lower amount and/or at a different time.

In some embodiments, the inhibitor can be an inhibitory nucleic acid; an aptamer; an antibody reagent; an antibody; or a small molecule. In some embodiments, the inhibitor of a target can be an inhibitor specific for that target. An inhibitor specific for a given target can be an inhibitor which binds specifically to the target molecule.

In one aspect of any of the embodiments, provided herein is an ATP-binding small molecule (e.g., extracellular ATP-binding small molecule) or composition comprising one or more of:

In various embodiments, the present invention provides a compound selected from the group consisting of:

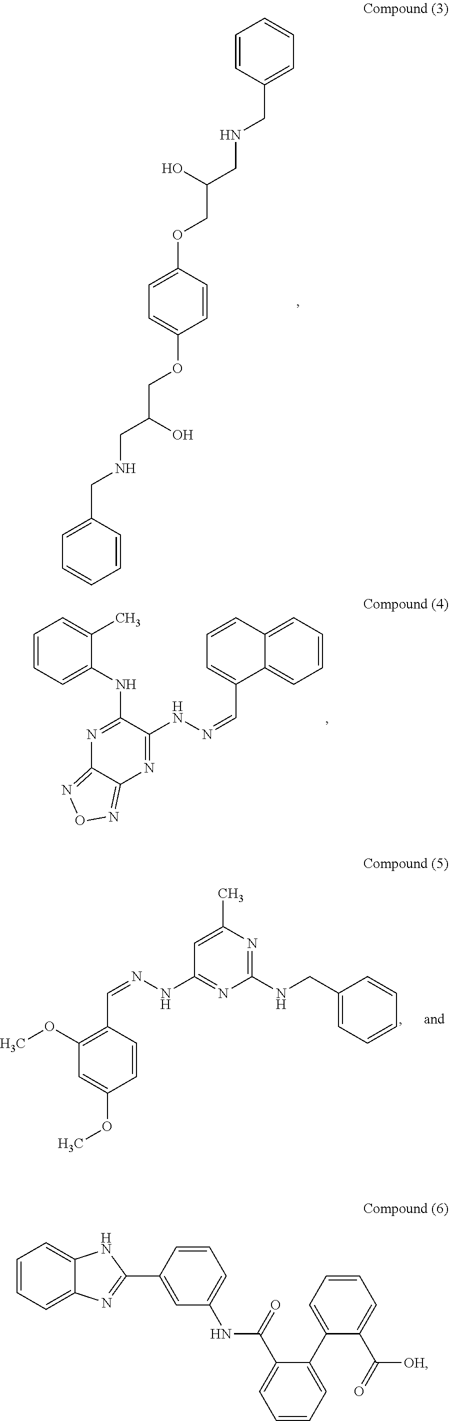

or a prodrug, isomer, enantiomer, diastereomer, racemate, tautomer, derivative, or salt thereof. The foregoing are referred to collectively herein as “ATP-binding small molecules.”

In various embodiments, the present invention provides a compound selected from the group consisting of:

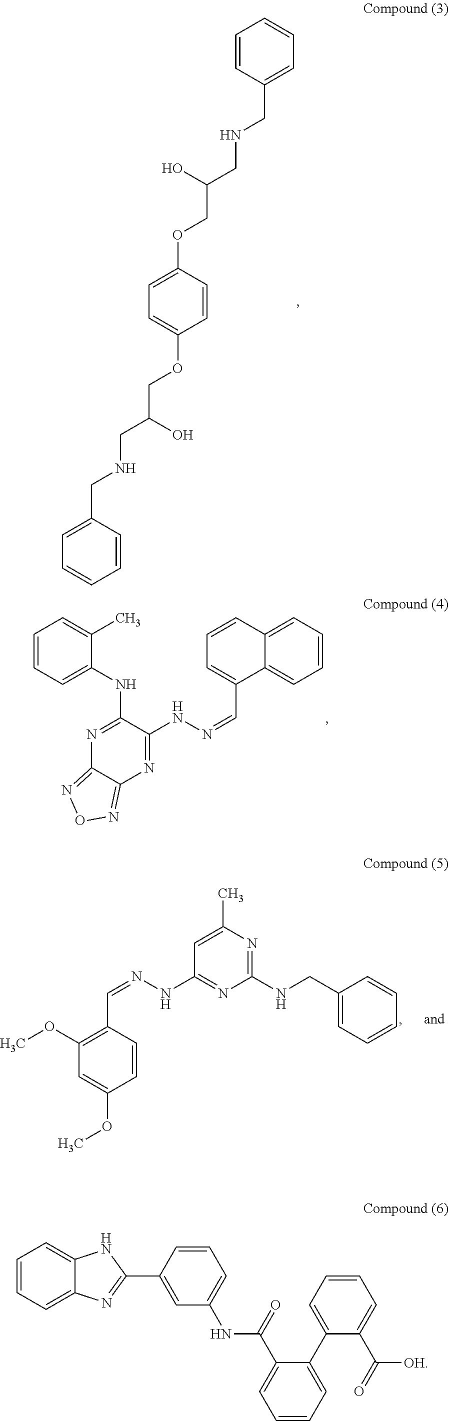

In various embodiments, the present invention provides a compound having the structure:

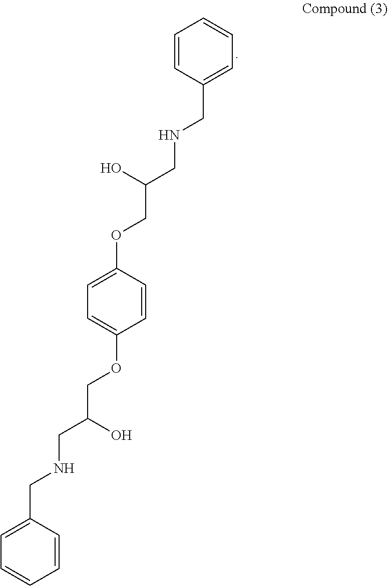 [SMILES: OC(CNCc1ccccc1)COc1ccc(OCC(O)CNCc2ccccc2)cc1]

In various embodiments, the present invention provides a compound having the structure:

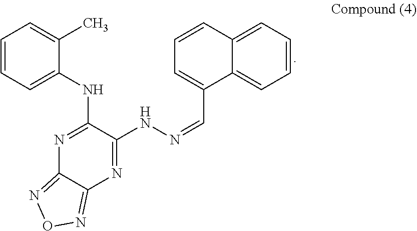 [SMILES: Cc1ccccc1Nc1nc2nonc2nc1N/N=C\c1cccc2ccccc12]

In various embodiments, the present invention provides a compound having the structure:

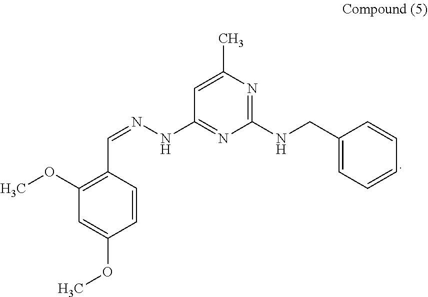 [SMILES: COc1ccc(/C=N\Nc2cc(C)nc(NCc3ccccc3)n2)c(OC)c1]

In various embodiments, the present invention provides a compound having the structure:

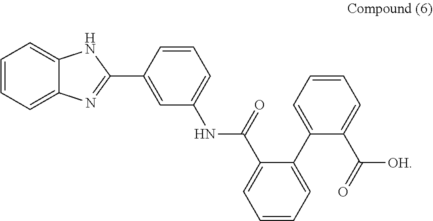 [SMILES: O=C(O)c1ccccc1-c1ccccc1C(=O)Nc1cccc(-c2nc3ccccc3[nH]2)c1]

In various embodiments, the present invention provides a composition comprising at least one compound selected from the group consisting of:

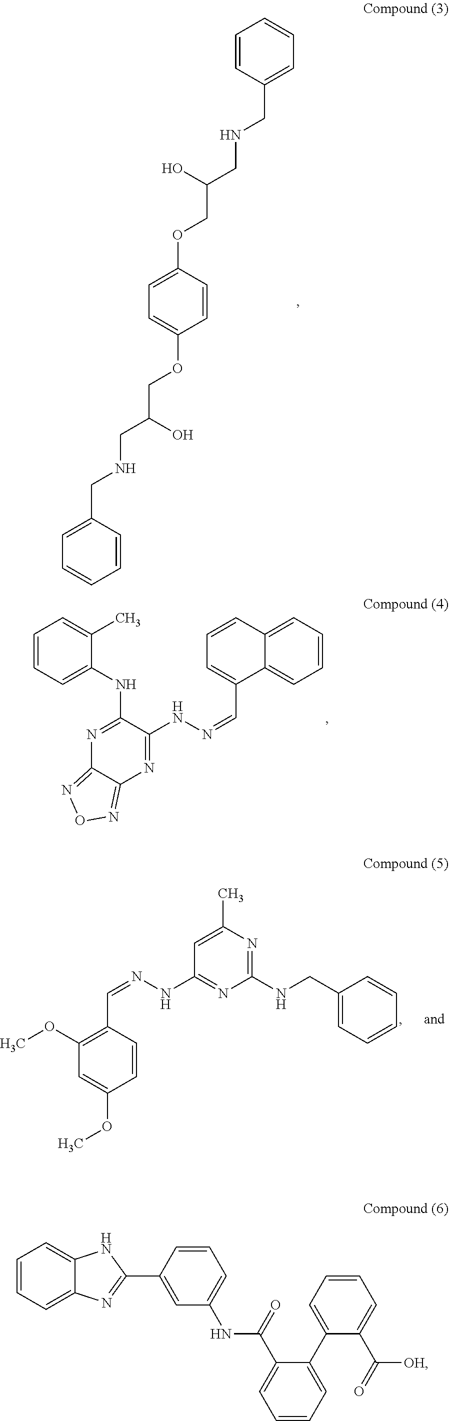

or a prodrug, isomer, enantiomer, diastereomer, racemate, tautomer, derivative, or salt thereof.

In various embodiments, the present invention provides a composition comprising at least one compound selected from the group consisting of:

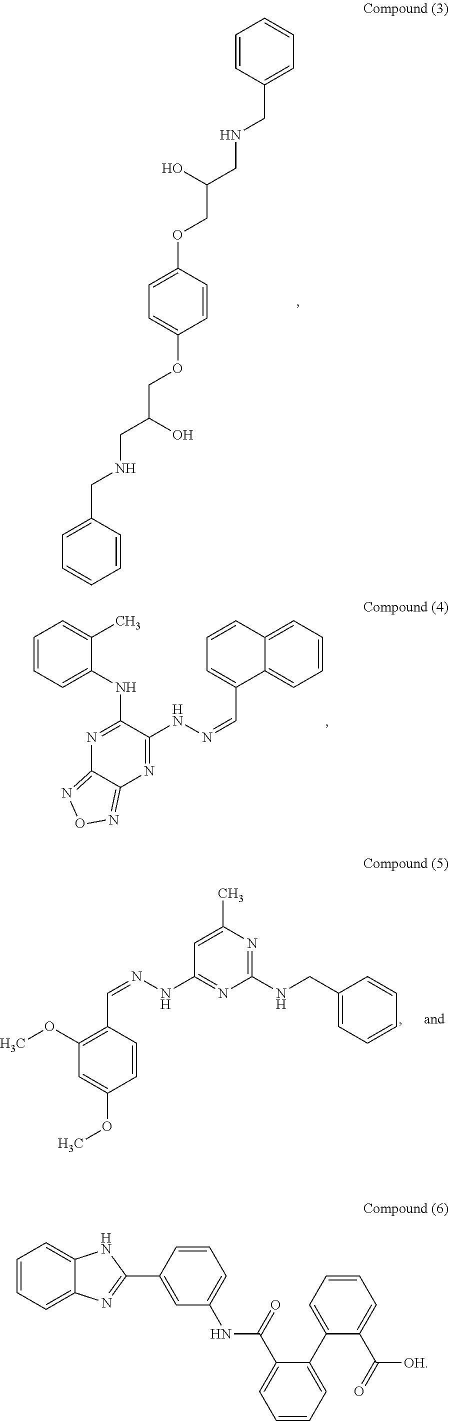

In various embodiments, the present invention provides a composition comprising:

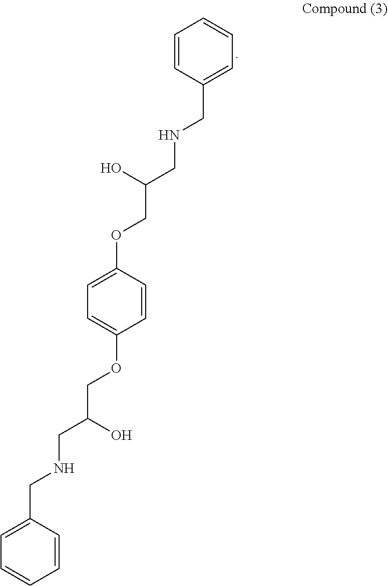 [SMILES: OC(CNCc1ccccc1)COc1ccc(OCC(O)CNCc2ccccc2)cc1]

In various embodiments, the present invention provides a composition comprising:

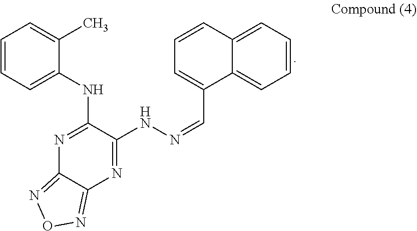 [SMILES: Cc1ccccc1Nc1nc2nonc2nc1N/N=C\c1cccc2ccccc12]

In various embodiments, the present invention provides a composition comprising:

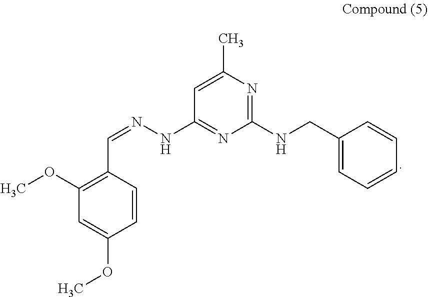 [SMILES: COc1ccc(/C=N\Nc2cc(C)nc(NCc3ccccc3)n2)c(OC)c1]

In various embodiments, the present invention provides a composition comprising:

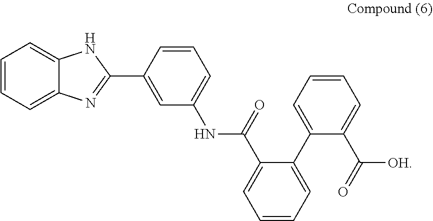 [SMILES: O=C(O)c1ccccc1-c1ccccc1C(=O)Nc1cccc(-c2nc3ccccc3[nH]2)c1]

In some embodiments, Compound (3) is selected from the group consisting of:

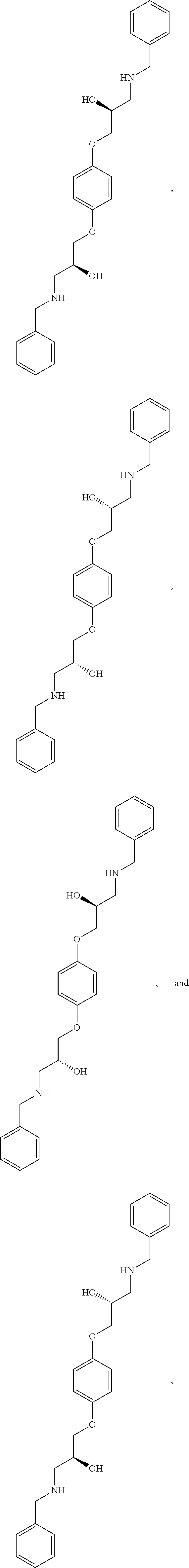 [SMILES: O[C@@H](CNCc1ccccc1)COc1ccc(OC[C@@H](O)CNCc2ccccc2)cc1 ; O[C@H](CNCc1ccccc1)COc1ccc(OC[C@H](O)CNCc2ccccc2)cc1 ; O[C@H](CNCc1ccccc1)COc1ccc(OC[C@@H](O)CNCc2ccccc2)cc1 ; O[C@H](CNCc1ccccc1)COc1ccc(OC[C@@H](O)CNCc2ccccc2)cc1]

or combinations thereof.

In some embodiments, the composition is a pharmaceutical composition.

In some embodiments, the salt is a pharmaceutically acceptable salt.

In some embodiments, the pharmaceutical composition comprises a pharmaceutically acceptable carrier.

In some embodiments, the at least one compound selected from the group consisting of:

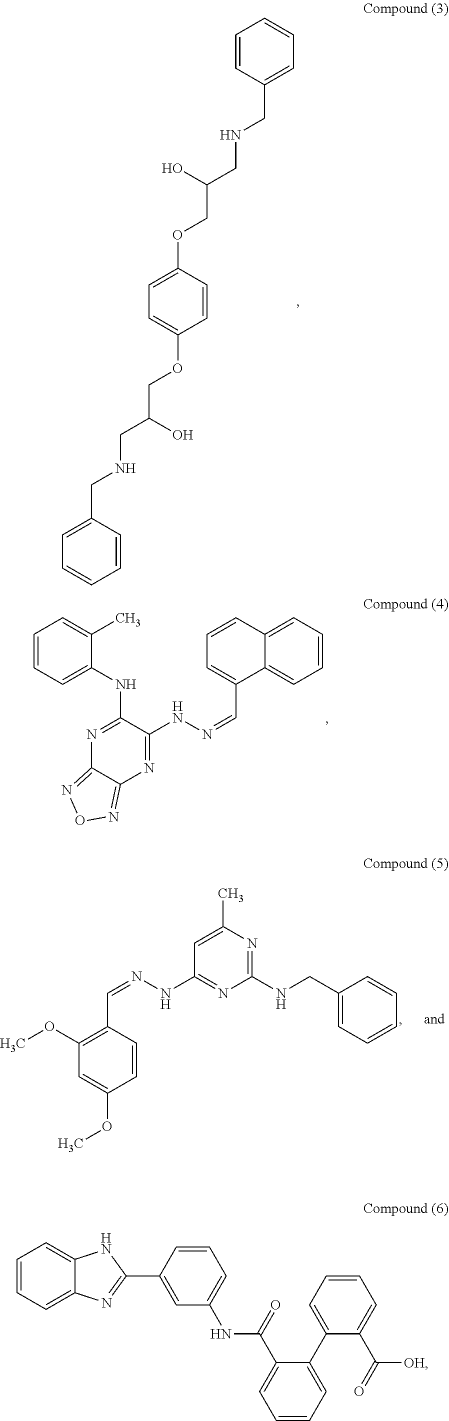

or a prodrug, isomer, enantiomer, diastereomer, racemate, tautomer, derivative, or salt thereof, is present in the composition in a therapeutically effective amount.

In some embodiments, the at least one compound selected from the group consisting of:

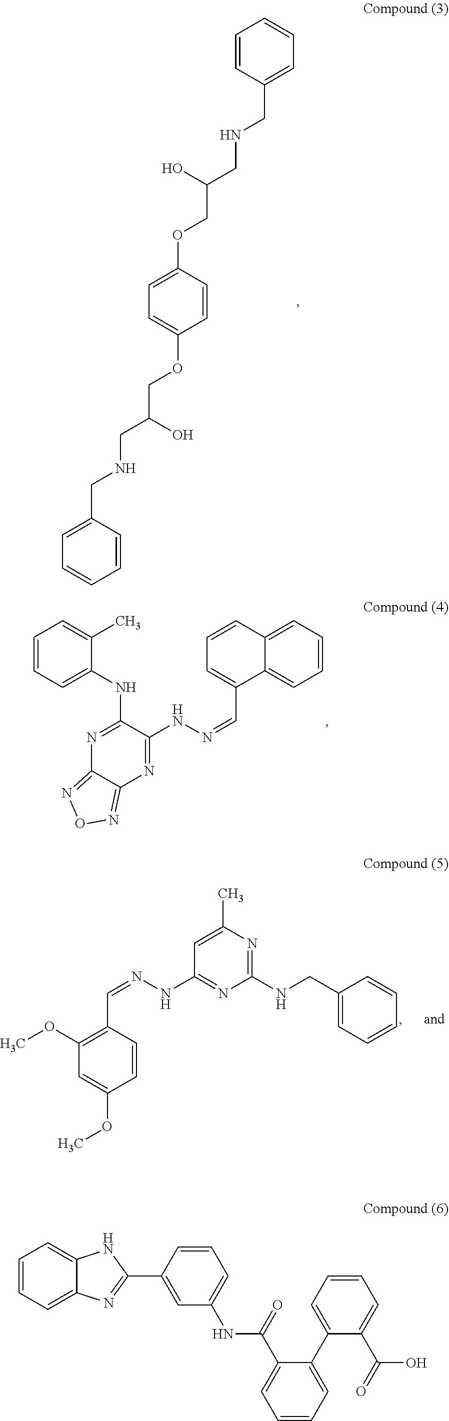

is present in the composition in a therapeutically effective amount.

In some embodiments,

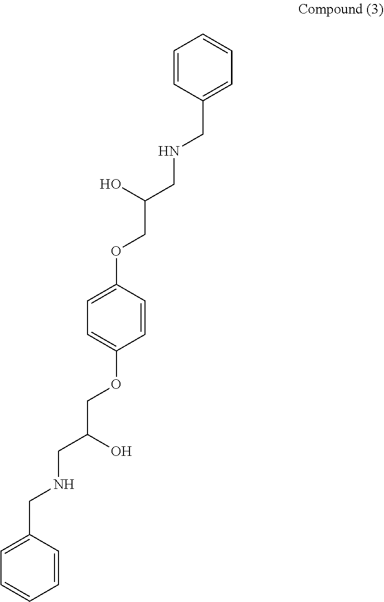 [SMILES: OC(CNCc1ccccc1)COc1ccc(OCC(O)CNCc2ccccc2)cc1]

is present in the composition in a therapeutically effective amount.

In some embodiments,

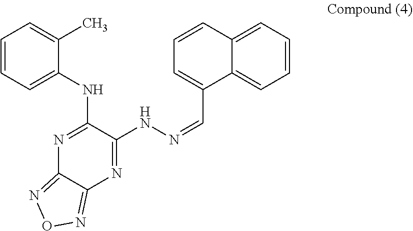 [SMILES: Cc1ccccc1Nc1nc2nonc2nc1N/N=C\c1cccc2ccccc12]

is present in the composition in a therapeutically effective amount.

In some embodiments,

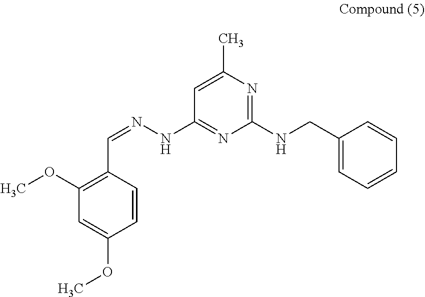 [SMILES: COc1ccc(/C=N\Nc2cc(C)nc(NCc3ccccc3)n2)c(OC)c1]

is present in the composition in a therapeutically effective amount.

In some embodiments,

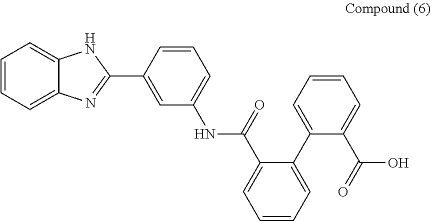 [SMILES: O=C(O)c1ccccc1-c1ccccc1C(=O)Nc1cccc(-c2nc3ccccc3[nH]2)c1]

is present in the composition in a therapeutically effective amount.

The term “prodrug” as used herein refers to compounds that can be converted via some chemical or physiological process (e.g., enzymatic processes and metabolic hydrolysis) to compound described herein. Thus, the term “prodrug” also refers to a precursor of a biologically active compound that is pharmaceutically acceptable. A prodrug can be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. For example, a compound comprising a hydroxy group can be administered as an ester that is converted by hydrolysis in vivo to the hydroxy compound. Suitable esters that can be converted in vivo into hydroxy compounds include acetates, citrates, lactates, tartrates, malonates, oxalates, salicylates, propionates, succinates, fumarates, formates, benzoates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group can be administered as an amide, e.g., acetamide, formamide and benzamide that is converted by hydrolysis in vivo to the amine compound. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5 (4): 265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. ll: 345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15 (2): 143-53 (1990); Balimane and Sinko, “Involvement of multiple transporters in the oral absorption of nucleoside analogues”, Adv. Drug Delivery Rev., 39 (1-3): 183-209 (1999); Browne, “Fosphenytoin (Cerebyx)”, Clin. Neuropharmacol. 20 (1): 1-12 (1997); Bundgaard, “Bioreversible derivatization of drugs-principle and applicability to improve the therapeutic effects of drugs”, Arch. Pharm. Chemi 86 (1): 1-39 (1979); Bundgaard H. “Improved drug delivery by the prodrug approach”, Controlled Drug Delivery 17:179-96 (1987); Bundgaard H. “Prodrugs as a means to improve the delivery of peptide drugs”, Arfv. Drug Delivery Rev. 8 (1): 1-38 (1992); Fleisher et al. “Improved oral drug delivery: solubility limitations overcome by the use of prodrugs”, Arfv. Drug Delivery Rev. 19 (2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72 (3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4:49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45 (6): 866-94 (1993); Sinhababu and Thakker, “Prodrugs of anticancer agents”, Adv. Drug Delivery Rev. 19 (2): 241-273 (1996); Stella et al., “Prodrugs. Do they have advantages in clinical practice?”, Drugs 29 (5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39 (1-3): 117-151 (1999); Taylor, “Improved passive oral drug delivery via prodrugs”, Adv. Drug Delivery Rev., 19 (2): 131-148 (1996); Valentino and Borchardt, “Prodrug strategies to enhance the intestinal absorption of peptides”, Drug Discovery Today 2 (4): 148-155 (1997); Wiebe and Knaus, “Concepts for the design of anti-HIV nucleoside prodrugs for treating cephalic HIV infection”, Adv. Drug Delivery Rev.: 39 (1-3): 63-80 (1999); Waller et al., “Prodrugs”, Br. J. Clin. Pharmac. 28:497-507 (1989), content of all of which are herein incorporated by reference in its entirety.

A “pharmaceutically acceptable salt”, as used herein, is intended to encompass any compound described herein that is utilized in the form of a salt thereof, especially where the salt confers on the compound improved pharmacokinetic properties as compared to the free form of compound or a different salt form of the compound. The pharmaceutically acceptable salt form can also initially confer desirable pharmacokinetic properties on the compound that it did not previously possess, and may even positively affect the pharmacodynamics of the compound with respect to its therapeutic activity in the body. An example of a pharmacokinetic property that can be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

Pharmaceutically acceptable salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), the content of which is herein incorporated by reference in its entirety. Exemplary salts also include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. Suitable acids which are capable of forming salts with the compounds of the disclosure include inorganic acids such as hydrochloric acid, hydrobromic acid, perchloric acid, nitric acid, thiocyanic acid, sulfuric acid, phosphoric acid, and the like; and organic acids such as 1,2-ethanedisulfonic acid, 2-hydroxyethanesulfonic acid, 2-naphthalenesulfonic acid, 3-phenylpropionic acid, 4-methylbicyclo[2.2.2]oct-2-ene-1-carboxylic acid, 4,4′-methylenebis(3-hydroxy-2-ene-1-carboxylic acid), acetic acid, anthranilic acid, benzenesulfonic acid, benzoic acid, camphorsulfonic acid, cinnamic acid, citric acid, cyclopentanepropionic acid, ethanesulfonic acid, formic acid, fumaric acid, glucoheptonic acid, gluconic acid, glutamic acid, glycolic acid, heptanoic acid, hydroxynaphthoic acid, lactic acid, lauryl sulfuric acid, maleic acid, malic acid, malonic acid, mandelic acid, methanesulfonic acid, muconic acid, naphthalene sulfonic acid, o-(4-hydroxybenzoyl)benzoic acid, oxalic acid, p-chlorobenzenesulfonic acid, propionic acid, p-toluenesulfonic acid, pyruvic acid, salicylic acid, stearic acid, succinic acid, sulfanilic acid, tartaric acid, tertiary butylacetic acid, trifluoroacetic acid, trimethylacetic acid, and the like. Suitable bases capable of forming salts with the compounds of the disclosure include inorganic bases such as sodium hydroxide, ammonium hydroxide, sodium carbonate, calcium hydroxide, potassium hydroxide and the like; and organic bases such as mono-, di- and tri-alkyl and aryl amines (e.g., triethylamine, diisopropyl amine, methyl amine, dimethyl amine, N-methylglucamine, pyridine, picoline, dicyclohexylamine, N,N′-dibezylethylenediamine, and the like), and optionally substituted ethanol-amines (e.g., ethanolamine, diethanolamine, trierhanolamine and the like).

In one aspect of any of the embodiments, described herein is a composition or combination comprising a) one or more ATP-binding small molecules, e.g., one ore more of compounds 3-6 and b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF).

As used herein “combination” refers to a group of two or more substances for use together, e.g., for administration to the same subject. The two or more substances can be present in the same formulation in any molecular or physical arrangement, e.g, in an admixture, in a solution, in a mixture, in a suspension, in a colloid, in an emulsion. The formulation can be a homogeneous or heterogenous mixture. In some embodiments of any of the aspects, the two or more substances active compound(s) can be comprised by the same or different superstructures, e.g., nanoparticles, liposomes, vectors, cells, scaffolds, or the like, and said superstructure is in solution, mixture, admixture, suspension with a solvent, carrier, or some of the two or more substances. Alternatively, the two or more substances can be present in two or more separate formulations, e.g., in a kit or package comprising multiple formulations in separate containers, to be administered to the same subject.

A kit is an assemblage of materials or components, including at least one composition or combination described herein. The exact nature of the components configured in the kit depends on its intended purpose. In some embodiments of any of the aspects, a kit includes instructions for use. “Instructions for use” typically include a tangible expression describing the technique to be employed in using the components of the kit, e.g., to treat a subject or for administration to a subject. Still in accordance with the present invention, “instructions for use” may include a tangible expression describing the preparation of composition or combination, such as dilution, mixing, or incubation instructions, and the like, typically for an intended purpose. Optionally, the kit also contains other useful components, such as, measuring tools, diluents, buffers, syringes, pharmaceutically acceptable carriers, or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging may also preferably provide an environment that protects from light, humidity, and oxygen. As used herein, the term “package” refers to a suitable solid matrix or material such as glass, plastic, paper, foil, polyester (such as polyethylene terephthalate, or Mylar) and the like, capable of holding the individual kit components. Thus, for example, a package can be a glass vial used to contain suitable quantities of a composition or combination described herein. The packaging material generally has an external label which indicates the contents and/or purpose of the kit and/or its components.

CE-224535 is 2-(4-Chloro-3-(3-(1-hydroxycycloheptyl) propanoyl)phenyl)-4-((2R)-2-hydroxy-3-methoxy-propyl)-1,2,4-triazine-3,5-dione, having the following structure:

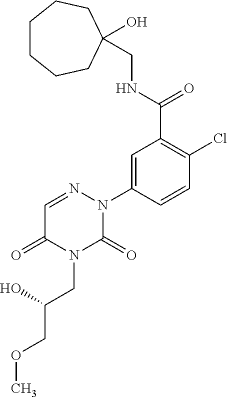 [SMILES: COC[C@H](O)Cn1c(=O)cnn(-c2ccc(Cl)c(C(=O)NCC3(O)CCCCCC3)c2)c1=O]

AZD9056 is N-(1-adamantylmethyl)-2-chloro-5-[3-(3-hydroxypropylamino) propyl]benzamide, having the following structure:

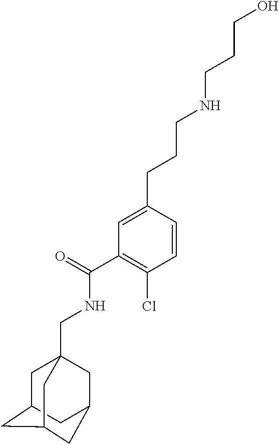 [SMILES: O=C(NCC12CC3CC(CC(C3)C1)C2)c1cc(CCCNCCCO)ccc1Cl]

GSK1482160 refers to (2S)—N-[[2-chloro-3-(trifluoromethyl)phenyl]methyl]-1-methyl-5-oxopyrrolidine-2-carboxamide, having the following structure:

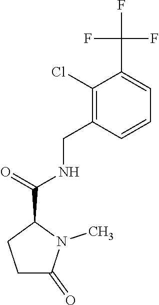 [SMILES: CN1C(=O)CC[C@H]1C(=O)NCc1cccc(C(F)(F)F)c1Cl]

Teplizumab is an anti-CD3 monoclonal antibody (also known as PRV-031, MGA031, and hOKT3γ1 (Ala-Ala)) produced by Provention Bio.

Rituximab is a anti-CD20 monoclonal antibody (also known as PRV-031, MGA031, and hOKT3γ1 (Ala-Ala)) and available commercially as RITUXAN from Genentech.

As used herein, the term “steroid” refers to a chemical substance comprising three cyclohexane rings and a cyclopentane ring. The rings are arranged to form tetracyclic cyclopentaphenanthrene, i.e. gonane. In some embodiments, the steroid is a corticosteroid.

As used herein, the term “corticosteroid” refers to a class of steroid hormones that are produced in the adrenal cortex or produced synthetically. Corticosteroids are involved in a wide range of physiologic systems such as stress response, immune response and regulation of inflammation, carbohydrate metabolism, protein catabolismblood electrolyte levels, and behavior. Corticosteroids are generally grouped into four classes, based on chemical structure. Group A corticosteroids (short to medium acting glucocorticoids) include hydrocortisone, hydrocortisone acetate, cortisone acetate, tixocortol pivalate, prednisolone, methylprednisolone, and prednisone. Group B corticosteroids include triamcinolone acetonide, triamcinolone alcohol, mometasone, amcinonide, budesonide, desonide, fluocinonide, fluocinolone acetonide, and halcinonide. Group C corticosteroids include betamethasone, betamethasone sodium phosphate, dexamethasone, dexamethasone sodium phosphate, and fluocortolone. Group D corticosteroids include hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate. Non-limiting examples of corticosteroids include, aldosternone, beclomethasone, beclomethasone dipropionate, betametahasone, betametahasone-21-phosphate disodium, betametahasone valerate, budesonide, clobetasol, clobetasol propionate, clobetasone butyrate, clocortolone pivalate, cortisol, cortisteron, cortisone, deflazacort, dexamethasone, dexamethasone acetate, dexamethasone sodium phosphate, diflorasone diacetate, dihydroxycortison, flucinonide, fludrocortisones acetate, flumethasone, flunisolide, flucionolone acetonide, fluticasone furate, fluticasone propionate, halcinonide, halpmetasone, hydrocortisone, hydrocortisone acetate, hydrocortisone succinate, 16α-hydroxyprednisolone, isoflupredone acetate, medrysone, methylprednisolone, prednacinolone, predricarbate, prednisolone, prednisolone acetate, prednisolone sodium succinate, prednisone, triamcinolone, triamcinolone, and triamcinolone diacetate. As used herein, the term “corticosteroid” can include, but is not limited to, the following generic and brand name corticosteroids: cortisone (CORTONE™ ACETATE™, ADRESON™, ALTESONA™, CORTELAN™, CORTISTAB™, CORTISYL™, CORTOGEN™, CORTONE™, SCHEROSON™); dexamethasone-oral (DECADRON-ORAL™, DEXAMETH™, DEXONE™, HEXADROL-ORAL™, DEXAMETHASONE™ INTENSOL™, DEXONE 0.5™, DEXONE 0.75™, DEXONE 1.5™, DEXONE 4™); hydrocortisone-oral (CORTEF™, HYDROCORTONE™); hydrocortisone cypionate (CORTEF ORAL SUSPENSION™); methylprednisolone-oral (MEDROL-ORAL™); prednisolone-oral (PRELONE™, DELTA-CORTEF™, PEDIAPRED™, ADNISOLONE™, CORTALONE™, DELTACORTRIL™, DELTASOLONE™, DELTASTAB™, DI-ADRESON F™, ENCORTOLONE™, HYDROCORTANCYL™, MEDISOLONE™, METICORTELONE™, OPREDSONE™, PANAAFCORTELONE™, PRECORTISYL™, PRENISOLONA™, SCHERISOLONA™, SCHERISOLONE™); prednisone (DELTASONE™, LIQUID PRED™, METICORTEN™, ORASONE 1™, ORASONE 5™, ORASONE 10™, ORASONE 20™, ORASONE 50™, PREDNICEN-M™, PREDNISONE INTENSOL™, STERAPRED™, STERAPRED DS™, ADASONE™, CARTANCYL™, COLISONE™, CORDROL™, CORTAN™, DACORTIN™, DECORTIN™, DECORTISYL™, DELCORTIN™, DELLACORT™, DELTADOME™, DELTACORTENE™, DELTISONA™, DIADRESON™, ECONOSONE™, ENCORTON™, FERNISONE™, NISONA™, NOVOPREDNISONE™ PANAFCORT™, PANASOL™, PARACORT™, PARMENISON™, PEHACORT™, PREDELTIN™, PREDNICORT™, PREDNICOT™, PREDNIDIB™, PREDNIMENT™, RECTODELT™, ULTRACORTEN™, WINPRED™); triamcinoloneoral (KENACORT™ ARISTOCORT™, ATOLONE™, SHOLOG ATM, TRAMACORT-D™, TRI-MED™ TRIAMCOT™, TRISTOPLEX™, TRYLONE D™, U-TRI-LONE™). Methods of synthesizing steroids and corticosteroids are well known in the art and such compounds are also commercially available.

Anti-Thymocyte Globulin (ATG) is a composition comprising anti-T cell and/or anti-thymocyte antibodies. The antibodies are often horse or rabbit antibodies. ATG is available commercially ast rATG (THYMOGLOBULIN™) from Genzyme, or equine ATG (ATGAM™) from Pfizer.

Mycophenolate mofetil (MMF) is 2-morpholin-4-ylethyl (E)-6-(4-hydroxy-6-methoxy-7-methyl-3-oxo-1H-2-benzofuran-5-yl)-4-methylhex-4-enoate, having the structure below. MMF is available commercially as CELLCEPT from Genentech.

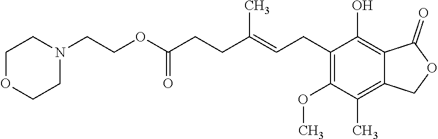 [SMILES: COc1c(C)c2c(c(O)c1C/C=C(\C)CCC(=O)OCCN1CCOCC1)C(=O)OC2]

Oxidized ATP (oATP) is an selective inhibitor of the P2Z/P2X7 ATP receptor, which is the 2′,3′-dialdehyde derivative of ATP.

As used herein, “transplantation cells” refer to cells of any cell type which are heterologous to the patient they are be administered to. The cells can be individual cells or a tissue. In some embodiments of any of the aspects, the cells are beta cells or islet cells.

It is contemplated herein that a composition or combination as described herein can comprise any two agents, any three agents, any four agents or more agents in any set, pairing, or combination. By way of non-limting example, a composition or combination can comprise multiple ATP-binding small molecules, e.g.:
A) Compound 3 and Compound 4;
B) Compound 3 and Compound 5;
C) Compound 3 and Compound 6;
D) Compound 4 and Compound 5;
E) Compound 4 and Compound 6;
F) Compound 5 and Compound 6;
G) Compound 3, Compound 4; and Compound 5;
H) Compound 3, Compound 4; and Compound 6;
I) Compound 3, Compound 5; and Compound 6;
J) Compound 4, Compound 5; and Compound 6; or
K) Compound 3, Compound 4; Compound 5; and Compound 6.

By way of further non-limiting example, a composition or combination can comprise any of the foregoing sets of ATP-binding small molecules paired with any of the additional agents described herein, e.g.:
a) one or more ATP-binding small molecules described herein and a population of transplantation cells,
b) one or more ATP-binding small molecules described herein and rapamycin,
c) one or more ATP-binding small molecules described herein and oATP,
d) one or more ATP-binding small molecules described herein and CE-224535,
e) one or more ATP-binding small molecules described herein and AZD9056,
f) one or more ATP-binding small molecules described herein and GSK1482160,
g) one or more ATP-binding small molecules described herein and a polypeptide comprising the ectodomain of P2X7R,
h) one or more ATP-binding small molecules described herein and a P2X7R soluble protein,
i) one or more ATP-binding small molecules described herein and a steroid,
j) one or more ATP-binding small molecules described herein and teplizumab,
k) one or more ATP-binding small molecules described herein and rituximab,
l) one or more ATP-binding small molecules described herein and anti-thymocyte globulin (ATG), or
m) one or more ATP-binding small molecules described herein and mycophenolate mofetil (MMF).

By way of further non-limiting example, a composition or combination can comprise any set or pairing of the one or more agents selected from: a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF). As illustrative examples, the following pairwise combinations are contemplated, with or without at least one ATP-binding small molecule.

TABLE 2
Exemplary pairwise sets

| | | | | | | | A |
|---|---|---|---|---|---|---|---|
| | A | | | | | | polypeptide |
| | population | | | | | | comprising |
| | of | | | | | | the |
| | transplantation | | | | | | ectodomain |
| | cells | Rapamycin | oATP | CE-224535 | AZD9056 | GSK1482160 | of P2X7R |
| A population of | | X | X | X | X | X | x |
| transplantation | | | | | | | |
| cells | | | | | | | |
| Rapamycin | x | | X | X | X | X | X |
| oATP | X | X | | X | X | X | X |
| CE-224535 | X | X | X | | X | X | |
| AZD9056 | X | X | X | X | | | X |
| GSK1482160 | X | X | X | X | X | X | X |
| A polypeptide | X | X | X | X | X | X | |
| comprising the | | | | | | | |
| ectodomain of | | | | | | | |
| P2X7R | | | | | | | |
| A P2X7R soluble | X | X | X | X | X | X | X |
| protein | | | | | | | |
| A steroid | X | X | X | X | X | X | X |
| Teplizumab | X | X | X | X | X | X | X |
| Rituximab | X | X | X | X | X | X | X |
| ATG | X | X | X | X | X | X | X |
| MMF | X | x | x | x | x | x | x |

| | A | | | | | |
|---|---|---|---|---|---|---|
| | P2X7R | | | | | |
| | soluble | A | | | | |
| | protein | Steroid | Teplizumab | Rituximab | ATG | MMF |
| A population of | X | X | x | X | x | X |
| transplantation | | | | | | |
| cells | | | | | | |
| Rapamycin | X | X | X | X | X | X |
| oATP | X | X | X | X | X | X |
| CE-224535 | X | X | X | X | X | X |
| AZD9056 | X | X | X | X | X | X |
| GSK1482160 | X | X | X | X | X | X |
| A polypeptide | X | X | X | X | X | X |
| comprising the | | | | | | |
| ectodomain of | | | | | | |
| P2X7R | | | | | | |
| A P2X7R soluble | | X | X | X | X | X |
| protein | | | | | | |
| A steroid | X | | X | X | X | X |
| Teplizumab | X | X | | X | X | X |
| Rituximab | X | X | X | | X | X |
| ATG | X | X | X | X | | x |
| MMF | x | x | x | x | x | |

In some embodiments, the technology described herein relates to a pharmaceutical composition comprising a) one or more ATP-binding small molecules, and/or b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) as described herein, and optionally a pharmaceutically acceptable carrier.

In some embodiments, the active ingredients of the pharmaceutical composition comprise a) one or more ATP-binding small molecules, and b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) as described herein. In some embodiments, the active ingredients of the pharmaceutical composition consist essentially of comprise a) one or more ATP-binding small molecules, and b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) as described herein. In some embodiments, the active ingredients of the pharmaceutical composition consist of comprise a) one or more ATP-binding small molecules, and b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) as described herein.

In some embodiments, the active ingredients of the pharmaceutical composition comprise one or more ATP-binding small molecules as described herein. In some embodiments, the active ingredients of the pharmaceutical composition consist essentially of one or more ATP-binding small molecules as described herein. In some embodiments, the active ingredients of the pharmaceutical composition consist of one or more ATP-binding small molecules as described herein.

In some embodiments, the active ingredients of the pharmaceutical composition comprise one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) as described herein. In some embodiments, the active ingredients of the pharmaceutical composition consist essentially of one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) as described herein. In some embodiments, the active ingredients of the pharmaceutical composition consist of one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) as described herein.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. a) one or more ATP-binding small molecules, and/or b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) as described herein.

The skilled practitioner can determine the appropriate dosage of the compounds described herein for an individual. In some embodiments of any of the aspects, a compound described herein can be administered at the following dosage:
CTLA-4-Ig (300-700 ug as induction at day 0, then 4 injection of 100-400 ug every 2 days following transplant, via intraperitoneal route),
oATP (100-400 ug/day for 14 days, via intraperitoneal route),
CE-224535 (100-400 ug/day for 14 days, via intraperitoneal route),
AZD9056 (100-400 ug/day for 28 days, via intraperitoneal route),
GSK1482160 (5-40 mg/kg twice a day for 30 days, oral gavage),
a polypeptide comprising the ectodomain of P2X7R (150-450 ul of PBS containing 10-500 ug of purified P2X7R, injected intravenous route),
a P2X7R soluble protein (300-700 ug as induction at day 0, then 100-400 ug at days 2, 4, 6, 8 and 10, via intraperitoneal route),
a steroid (daily injection of 0.01-1.0 mg/kg (7 days), via intraperitoneal route),
teplizumab (1-10 ug/day for 5 days, intravenous injection),
rituximab (100-400 μg per day for 7 days, via intraperitoneal route),
anti-thymocyte globulin (ATG) (300-700 ug as induction at day 0, then 4 injections of 100-400 ug every 2 days following transplant, via intraperitoneal route),
mycophenolate mofetil (MMF) (10-100 mg/kg administered twice daily (b.i.d.)
subcutaneously for 7 days and subsequently administered by oral gavage BID), and/or
rapamycin (0.01-1.0 mg/day for 10 days, via intraperitoneal route).
   In some embodiments of any of the aspects, a compound described herein can be administered at the following dosage:
CTLA-4-Ig (500 ug as induction at day 0, then 4 injection of 250 ug every 2 days following transplant, via intraperitoneal route),
oATP (250 ug/day for 14 days, via intraperitoneal route),
CE-224535 (250 ug/day for 14 days, via intraperitoneal route),
AZD9056 (250 ug/day for 28 days, via intraperitoneal route),
GSK1482160 (20 mg/kg twice a day for 30 days, oral gavage),
a polypeptide comprising the ectodomain of P2X7R (300 ul of PBS containing 50 ug or 300 ug of purified P2X7R, injected intravenous route),
a P2X7R soluble protein (500 ug as induction at day 0, then 250 ug at days 2, 4, 6, 8 and 10, via intraperitoneal route),
a steroid (daily injection of 0.1 mg/kg (7 days), via intraperitoneal route),
teplizumab (5 ug/day for 5 days, intravenous injection),
rituximab (250 μg per day for 7 days, via intraperitoneal route),
anti-thymocyte globulin (ATG) (500 ug as induction at day 0, then 4 injections of 250 ug every 2 days following transplant, via intraperitoneal route),
mycophenolate mofetil (MMF) (50 mg/kg administered twice daily (b.i.d.) subcutaneously for 7 days and subsequently administered by oral gavage BID), and/or
rapamycin (0.1 mg/day for 10 days, via intraperitoneal route).

In some embodiments, the pharmaceutical composition comprising a) one or more ATP-binding small molecules, and/or b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a) one or more ATP-binding small molecules, and/or b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a) one or more ATP-binding small molecules, and/or b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) as disclosed herein can also be incorporated into the parenteral dosage forms of the disclosure, including conventional and controlled-release parenteral dosage forms.

Pharmaceutical compositions comprising a) one or more ATP-binding small molecules, and/or b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition(s) can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, the polypeptide, ATP-binding small molecule, or other agent present in a composition, or combination, of the disclosure exhibits an increased utility that is not exhibited when said polypeptide, ATP-binding small molecule, or other agent occurs alone or when said polypeptide, ATP-binding small molecule, or other agent are present at a naturally occurring concentration. In some embodiments of any of the aspects, compositions of the disclosure, comprising a polypeptide, ATP-binding small molecule, and/or other agent as taught herein, exhibit a synergistic effect on imparting at least one improved trait in an subject. In some embodiments of any of the aspects, the compositions of the disclosure—comprising a polypeptide, ATP-binding small molecule, or other agent as taught herein—exhibit markedly different characteristics/properties compared to their closest naturally occurring counterpart. That is, the compositions of the disclosure exhibit markedly different functional and/or structural characteristics/properties, as compared to their closest naturally occurring counterpart. For instance, the polypeptide, ATP-binding small molecule, or other agent of the disclosure are structurally different from a polypeptide, ATP-binding small molecule, or other agent as it naturally exists in a source cell or organism, for at least the following reasons: said polypeptide, ATP-binding small molecule, or other agent can be isolated and purified, such that it is not found in the milieu of the source cell or organism, said polypeptide, ATP-binding small molecule, or other agent can be present at concentrations that do not occur in the source cell or organism, said polypeptide, ATP-binding small molecule, or other agent can be associated with acceptable carriers that do not occur in the source cell or organism, said polypeptide, ATP-binding small molecule, or other agent can be formulated to be shelf-stable and exist outside the source cell or organism environment, and said polypeptide, ATP-binding small molecule, or other agent can be combined with other agents at concentrations that do not exist in the source cell or organism. Further, the polypeptide, ATP-binding small molecule, or other agent of the disclosure are functionally different from a polypeptide, ATP-binding small molecule, or other agent as it naturally exists in a source cell or organism, for at least the following reasons: said polypeptide, ATP-binding small molecule, or other agent when applied in an isolated and purified form can lead to inhibition of ATP/P2X7R signaling, said polypeptide, ATP-binding small molecule, or other agent can be formulated to be shelf-stable and able to exist outside the source cell or organism environment, such that the polypeptide, ATP-binding small molecule, or other agent now has a new utility as a supplement or compositions capable of administration to a subject, wherein the polypeptide, ATP-binding small molecule, or other agent could not have such a utility in its natural state in the source cell or organism, as the polypeptide, ATP-binding small molecule, or other agent would be unable to survive outside the source cell or organism without the intervention of the hand of man to formulate the polypeptide, ATP-binding small molecule, or other agent into a shelf-stable state and impart this new utility that has the aforementioned functional characteristics not possessed by the polypeptide, ATP-binding small molecule, or other agent in it's natural state of existence in the source cell or organism.

The interaction of ATP and P2X7R promotes phosphorylation of STAT3, Th1 and Th17 differentiation, and CD4+ proliferation and IFN-γ production. These processes then lead to adverse or pathological immunological responses. In particular, ATP/P2X7R signaling is known to cause, drive, or exacerbate immune-related diseases including type 1 diabetes, transplant rejection, or lung fibrosis. Accordingly, inhibition of ATP/P2X7R signaling, e.g., by administration of one of the ATP/P2X7R signaling inhibitors described herein can treat or prevent such immune-related diseases.

In one aspect of any of the embodiments, described herein is a method of treating or preventing an immune-related disease, type 1 diabetes, transplant rejection, or lung fibrosis in a subject in need thereof, the method comprising administering a composition or combination described herein, e.g., a) one or more ATP-binding small molecules, and/or b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF).

In some embodiments of any of the aspects, prevention or treatment can comprise slowing or inhibiting the onset or worsening of a disease or one or more symptoms of a disease. In some embodiments of any of the aspects, the treatment or prevention of type 1 diabetes comprises slowing the progression or onset of type 1 diabetes.

In some embodiments of any of the aspects, the subject has previously received a transplant of an organ and/or cells and the treatment or prevention of transplant rejection comprises suppressing or delaying rejection of the transplant. In some embodiments of any of the aspects, the subject is concurrently receiving a transplant of an organ and/or cells and the treatment or prevention of transplant rejection comprises suppressing or delaying rejection of the transplant. Suppressing a transplant rejection can refer to preventing one or more symptoms of rejection, decreasing one or more symptoms of rejection, reducing the need for other immune system inhibitors to be administered to the subject, and/or increasing the extent and/or time of transplant or graft success. The transplant or graft can be a tissue graft and/or an organ transplantation. In some embodiments, treating or preventing transplant rejection comprises delaying transplant rejection.

As used herein, the term “diabetes” refers a syndrome of disordered metabolism, usually due to a combination of hereditary and environmental causes, resulting in abnormally high blood sugar levels (hyperglycemia). Type 1 diabetes arises from a diminished production of insulin and leads to type to hyperglycemia, which largely causes the acute signs of diabetes: excessive urine production, resulting compensatory thirst and increased fluid intake, blurred vision, unexplained weight loss, lethargy, and changes in energy metabolism. Diabetes can cause many complications. Acute complications (hypoglycemia, ketoacidosis, or nonketotic hyperosmolar coma) may occur if the disease is not adequately controlled. Serious long-term complications (i.e. chronic side effects) include cardiovascular disease (doubled risk), chronic renal failure, retinal damage (which can lead to blindness), nerve damage (of several kinds), and microvascular damage, which may cause impotence and poor wound healing. Poor healing of wounds, particularly of the feet, can lead to gangrene, and possibly to amputation. In some embodiments, the diabetes can be type 1 diabetes. In some embodiments, a subject can be pre-diabetic, or susceptible to developing diabetes which can be characterized, for example, as having elevated fasting blood sugar, elevated post-prandial blood sugar, a family history of diabetes, impaired glucose tolerance, and/or impaired fasting glycaemia.

During the onset of type 1 diabetes (TID), T cells are fully activated at the immune synapse (IS) between pancreatic islets and T cells when autoantigens are presented by the TCR-MHC complex in the presence of an IL-2 signal and costimulation; however, other signals (e.g. inflammation) play a role in this process. Inhibition of ATP/2X7R signaling promotes islet graft survival, delays diabetes onset, induces donor-specific hyporesponsiveness, and inhibits allo- and autoimmune responses.

In general, lung fibrosis refers to a group of diseases associated with loss of lung functions due to a lesion regarding the reconstruction of an alveolar region, which is caused by the phenomenon whereby the alveolar structure is destroyed by an inflammatory reaction, and as a result, growth of fibroblasts and an excessive increase in extracellular matrix mainly composed of collagen take place, so that the lung becomes hardened. The term “pulmonary fibrosis” or “lung fibrosis” therefore refers to the formation or development of excessive fibrous connective tissue in the lung (fibrosis), thereby developing scar (fibrous) tissue.

The term “immune related disease” refers to a disease in which a component of the mammalian immune system causes, mediates or contributes to a mammal's morbidity. Also included are diseases in which stimulation or treatment of the immune response has an ameliorating effect on disease progression. The term includes immune-mediated pro-inflammatory diseases, non-immune-mediated pro-inflammatory diseases, infectious diseases, immunodeficiency diseases, tumorigenesis and the like.

ATP/P2X7R signaling is also known to cause, drive, or exacerbate aging process. Accordingly, inhibition of ATP/P2X7R signaling, e.g., by administration of one of the ATP/P2X7R signaling inhibitors described herein can slow or delay aging processes, or extend lifespan. Accordingly, in one of the aspects of any of the embodiments, described herein is a method of slowing or delaying aging processes or extending lifespan in a subject, the method comprising administering a composition or combination of any one of the preceding claims to the subject.

As demonstrated in the Examples herein, a subject having the rs3751143 SNP mutation in their P2X7R gene is more susceptible to activation of ATP/P2X7R signaling, including aberrant and/or pathological ATP/P2X7R signaling. In particular, this mutation increases the likelihood of the subject having or developing hyper-Th17 syndrome. Accordingly, in some embodiments of any of the aspects, the subject is a subject having the rs3751143 SNP mutation in P2X7R. In some embodiments of any of the aspects, the subject is a subject determined to have the rs3751143 SNP mutation in P2X7R. In some embodiments of any of the aspects, the subject is a subject having or determined to have hyper-Th17 syndrome.

In some embodiments of any of the aspects, the method comprises administering a composition or combination described herein to a subject previously determined to have the rs3751143 SNP mutation in P2X7R. In some embodiments of any of the aspects, described herein is a method of treating immune-related diseases, type 1 diabetes, transplant rejection, or lung fibrosis in a subject in need thereof, the method comprising: a) first detecting the presence or absence of the rs3751143 SNP mutation in P2X7R in a sample obtained from a subject; and b) then administering a composition or combination described herein to the subject if the SNP mutation is present.

In one aspect of any of the embodiments, described herein is a method of treating immune-related diseases, type 1 diabetes, transplant rejection, or lung fibrosis in a subject in need thereof, the method comprising: a) determining if the subject has detecting the presence or absence of the rs3751143 SNP mutation in P2X7R; and b) administering a composition or combination as described herein to the subject if SNP mutation is present. In some embodiments of any of the aspects, the step of determining if the subject has the rs3751143 SNP mutation in P2X7R can comprise i) obtaining or having obtained a sample from the subject and ii) performing or having performed an assay on the sample obtained from the subject to detect the presence or absence of the rs3751143 SNP mutation in P2X7R. In some embodiments of any of the aspects, the step of detecting if the subject has the SNP mutation can comprise performing or having performed an assay on a sample obtained from the subject to detect the presence or absence of the rs3751143 SNP mutation in P2X7R in the subject. In some embodiments of any of the aspects, the step of determining if the subject has the SNP mutation can comprise ordering or requesting an assay on a sample obtained from the subject to detect the presence or absence of the rs3751143 SNP mutation in P2X7R in the subject. In some embodiments of any of the aspects, the step of determining if the subject the SNP mutation can comprise receiving the results of an assay on a sample obtained from the subject to detect the presence or absence of the rs3751143 SNP mutation in P2X7R in the subject. In some embodiments of any of the aspects, the step of determining if the subject has the SNP mutation can comprise receiving a report, results, or other means of identifying the subject as a subject with the SNP mutation.

In one aspect of any of the embodiments, described herein is a method of treating immune-related diseases, type 1 diabetes, transplant rejection, or lung fibrosis in a subject in need thereof, the method comprising: a) determining if the subject has the rs3751143 SNP mutation in P2X7R; and b) instructing or directing that the subject be administered a composition or combination described herein if the SNP mutation is present. In some embodiments of any of the aspects, the step of determining if the subject has the SNP mutation can comprise i) obtaining or having obtained a sample from the subject and ii) performing or having performed an assay on the sample obtained from the subject to detect the presence or absence of the rs3751143 SNP mutation in P2X7R in the subject. In some embodiments of any of the aspects, the step of determining if the subject has the SNP mutation can comprise performing or having performed an assay on a sample obtained from the subject to detect the presence or absence of the rs3751143 SNP mutation in P2X7R in the subject. In some embodiments of any of the aspects, the step of determining if the subject has the SNP mutation can comprise ordering or requesting an assay on a sample obtained from the subject to detect the presence or absence of the rs3751143 SNP mutation in P2X7R in the subject. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results.

In some embodiments of any of the aspects, if the subject does not have the SNP mutation, they are administered (or instructions are provided to administer to them) an immunosuppression therapy selected from calcineurin inhibitors, mTOR inhibitors, or azathioprine. In some embodiments of any of the aspects, if the subject does not have the SNP mutation, they are administered (or instructions are provided to administer to them) an immunosuppression therapy selected from calcineurin inhibitors, mTOR inhibitors, steroids, MMF, or azathioprine.

Methods to detect SNPS are known to a skilled artisan. Such methods include PCR procedures, RT-PCR, quantitative RT-PCR Northern blot analysis, differential gene expression, RNAse protection assay, microarray based analysis, next-generation sequencing; hybridization methods, etc.

In some embodiments of any of the aspects, the sequence of no more than 200 other genes is determined. In some embodiments of any of the aspects, the sequence of no more than 100 other genes is determined. In some embodiments of any of the aspects, the sequence of no more than 20 other genes is determined. In some embodiments of any of the aspects, the sequence of no more than 10 other genes is determined.

The term “sample” or “test sample” as used herein denotes a sample taken or isolated from a biological organism, e.g., a blood or plasma sample from a subject. In some embodiments of any of the aspects, the present invention encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject.

The test sample can be obtained by removing a sample from a subject, but can also be accomplished by using a previously isolated sample (e.g. isolated at a prior timepoint and isolated by the same or another person).

In some embodiments of any of the aspects, the methods, assays, and systems described herein can further comprise a step of obtaining or having obtained a test sample from a subject. In some embodiments of any of the aspects, the subject can be a human subject. In some embodiments of any of the aspects, the subject can be a subject in need of treatment for (e.g. having or diagnosed as having) a condition described herein or a subject at risk of or at increased risk of developing a condition as described elsewhere herein.

In some embodiments, the methods described herein relate to treating a subject having or diagnosed as having a condition described herein with a) one or more ATP-binding small molecules, and/or b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF). Subjects having such conditions can be identified by a physician using current methods of diagnosing the relevant condition. Symptoms and/or complications of such conditions are known in the art.

The compositions and methods described herein can be administered to a subject having or diagnosed as having a condition described herein, e.g., diabetes. In some embodiments, the methods described herein comprise administering an effective amount of compositions described herein to a subject in order to alleviate a symptom of a condition described herein, e.g., diabetes. As used herein, “alleviating a symptom” is ameliorating any condition or symptom associated with the disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of a) one or more ATP-binding small molecules, and/or b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) needed to alleviate at least one or more symptom of the disease or disorder, and relates to a sufficient amount of pharmacological composition to provide the desired effect. The term “therapeutically effective amount” therefore refers to an amount of a) one or more ATP-binding small molecules, and/or b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) nthat is sufficient to provide a particular therapeutic or prophylactic effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of an active ingredient, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for islet cell survival or immune responses, among others. The dosage can be determined by a physician and adjusted, as necessary, to suit observed effects of the treatment.

In some embodiments of any of the aspects, the a) one or more ATP-binding small molecules, and/or b) one or more of a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF) described herein is administered as a monotherapy, e.g., another treatment for the disease or condition is not administered to the subject.

The methods described herein can further comprise administering a second agent and/or treatment to the subject, e.g. as part of a combinatorial therapy. By way of non-limiting example, if a subject is to be treated for pain or inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs-such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

In certain embodiments, an effective dose of a composition or combination as described herein can be administered to a patient once. In certain embodiments, an effective dose of a composition or combination described herein can be administered to a patient repeatedly. For systemic administration, subjects can be administered a therapeutic amount of a composition or combination as described herein, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient(s). The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition or combination as described herein can be administered over a period of time, such as over a 5 minute, 10 minute, 15 minute, 20 minute, or 25 minute period.

The dosage ranges for the administration of a composition or combination as described herein, according to the methods described herein depend upon, for example, the form of the active ingredient, its potency, and the extent to which symptoms, markers, or indicators of a condition described herein are desired to be reduced, for example the percentage reduction desired for immune responses or the extent to which, for example, islet cell survival are desired to be induced. The dosage should not be so large as to cause adverse side effects, such as detrimental immunosuppression. Generally, the dosage will vary with the age, condition, and sex of the patient and can be determined by one of skill in the art. The dosage can also be adjusted by the individual physician in the event of any complication.

The efficacy of a composition or combination described herein in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. immune responses or islet cell survial. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of animal models of diabetes. When using an experimental animal model, efficacy of treatment is evidenced when a statistically significant change in a marker is observed.

In one respect, the present invention relates to the herein described compositions, methods, and respective component(s) thereof, as essential to the technology, yet open to the inclusion of unspecified elements, essential or not (“comprising). In some embodiments of any of the aspects, other elements to be included in the description of the composition, method or respective component thereof are limited to those that do not materially affect the basic and novel characteristic(s) of the technology (e.g., the composition, method, or respective component thereof “consists essentially of” the elements described herein). This applies equally to steps within a described method as well as compositions and components therein. In other embodiments of any of the aspects, the compositions, methods, and respective components thereof, described herein are intended to be exclusive of any element not deemed an essential element to the component, composition or method (e.g., the composition, method, or respective component thereof “consists of” the elements described herein). This applies equally to steps within a described method as well as compositions and components therein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of conditions described herein, e.g., diabetes. A subject can be male or female.

A subject can be one who has been previously diagnosed with or identified as suffering from or having a condition in need of treatment (e.g. diabetes) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing. The terms also refer to fragments or variants of the polypeptide that maintain at least 50% of the activity or effect, e.g. ATP-binding ability, of the full length polypeptide. Conservative substitution variants that maintain the activity of wildtype P2X7R will include a conservative substitution as defined herein. The identification of amino acids most likely to be tolerant of conservative substitution while maintaining at least 50% of the activity of the wildtype is guided by, for example, sequence alignment with P2X7R homologs or paralogs from other species. Amino acids that are identical between P2X7R homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants, fragments, and/or fusion proteins can be tested for activity, for example, by administering the variant to an appropriate animal model of a condition as described herein.

In some embodiments, a polypeptide, e.g., a P2X7R polypeptide, can be a variant of a sequence described herein, e.g. a variant of a P2X7R polypeptide comprising the amino acid sequence of SEQ ID NO: 2 or 4. In some embodiments, the variant is a conservative substitution variant. Variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein, e.g., can bind ATP at least 50% as well as wildtype P2X7R. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of wildtype P2X7R, e.g. 110%, 125%, 150%, 175%, 200%, 500%, 1000% or more.

One method of identifying amino acid residues which can be substituted is to align, for example, human P2X7R to a P2X7R homolog from one or more non-human species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. The variant amino acid or DNA sequence can be at least 90%, at least 95%, at least 96%, at least 97%, at least 98%, at least 99%, or more, identical to a native or reference sequence, e.g. SEQ ID NO: 2 or 4 or a nucleic acid encoding one of those amino acid sequences. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp or BLASTn (available on the world wide web at blast.ncbi.nlm.nih.gov), with default parameters set.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. ATP-binding activity and specificity of a native or reference polypeptide is retained.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu. Typically conservative substitutions for one another also include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

In some embodiments, a polypeptide, e.g., a P2X7R polypeptide can comprise one or more amino acid substitutions or modifications. In some embodiments, the substitutions and/or modifications can prevent or reduce proteolytic degradation and/or prolong half-life of the polypeptide in a subject. In some embodiments, a polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov, 2002); and/or Fc fragments (Ashkenazi and Chamow, 1997). The references in the foregoing paragraph are incorporated by reference herein in their entireties.

In some embodiments, a polypeptide, e.g., a P2X7R polypeptide, as described herein can comprise at least one peptide bond replacement. A P2X7R polypeptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments, a polypeptide, e.g., a P2X7R polypeptide, as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser(S), Thr (T), Cys (C), Tyr (Y), Asn (N), Gln (Q), Asp (D), Glu (E), Lys (K), Arg (R), and His (H). In some embodiments, a P2X7R polypeptide as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, a polypeptide, e.g. a P2X7R polypeptide, can be modified, e.g. by addition of a moiety to one or more of the amino acids that together comprise the peptide. In some embodiments, a polypeptide as described herein can comprise one or more moiety molecules, e.g. 1 or more moiety molecules per polypeptide, 2 or more moiety molecules per polypeptide, 5 or more moiety molecules per polypeptide, 10 or more moiety molecules per polypeptide or more moiety molecules per polypeptide. In some embodiments, a polypeptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties. The half-life of a polypeptide can be increased by the addition of moieties, e.g. PEG, albumin, or other fusion partners (e.g. Fc fragment of an immunoglobin).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established. Alterations of the original amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Khudyakov et al. “Artificial DNA: Methods and Applications” CRC Press, 2002; Braman “In Vitro Mutagenesis Protocols” Springer, 2004; and Rapley “The Nucleic Acid Protocols Handbook” Springer 2000; which are herein incorporated by reference in their entireties. In some embodiments, a polypeptide as described herein can be chemically synthesized and mutations can be incorporated as part of the chemical synthesis process.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

In some embodiments, the methods described herein relate to measuring, detecting, or determining the level of at least one marker. As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, an agent described herein is exogenous. In some embodiments of any of the aspects, an agent described herein is ectopic. In some embodiments of any of the aspects, an agent described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. diabetes. The term “treating” includes reducing or alleviating at least one adverse effect or symptom of a condition, disease or disorder associated with a condition. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. Accordingly, the methods described herein can be prophylactic relative to onset of symptoms of diabetes or transplant rejection. In some embodiments of any of the aspects, prophylactic treatment is not prevention of all symptoms or signs of a disease.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “nanoparticle” refers to particles that are on the order of about 1 to 1,000 nanometers in diameter or width. The term “nanoparticle” includes nanospheres; nanorods; nanoshells; and nanoprisms; these nanoparticles may be part of a nanonetwork. The term “nanoparticles” also encompasses liposomes and lipid particles having the size of a nanoparticle. Exemplary nanoparticles include lipid nanoparticles or ferritin nanoparticles. Lipid nanoparticles can comprise multiple componenents, including, e.g., ionizable lipids (such as MC3, DLin-MC3-DMA, ALC-0315, or SM-102), pegylated lipids (such as PEG2000-C-DMG, PEG2000-DMG, ALC-0159), phospholipids (such as DSPC), and cholesterol.

Exemplary liposomes can comprise, e.g., DSPC, DPPC, DSPG, Cholesterol, hydrogenated soy phosphatidylcholine, soy phosphatidyl choline, methoxypolyethylene glycol (mPEG-DSPE) phosphatidyl choline (PC), phosphatidyl glycerol (PG), distearoylphosphatidylcholine, and combinations thereof.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

In all embodiments where a sample is obtained or has been obtained or provided, the sample can be sample taken, obtained, or provided via minimally invasive methods and/or involves only a minor intervention. In some embodiments of any of the aspects, a sample is taken, obtained, or provided by one or more of a blood draw or prick, an epidermal or mucus membrane swab, buccal sampling, saliva sample, a epidermal skin sampling technique, and/or collection of a secreted or expelled bodily fluid (e.g., mucus, urine, sweat, etc), fecal sampling, semen/seminal fluid sampling, or clippings (e.g., of hair or nails). In some embodiments of any of the aspects, the sample comprises, consists of, or consists essentially of blood (or any fraction or component thereof), serum, urine, mucus, epithelial cells, saliva, buccal cells, a secreted or expelled bodily fluid, and/or hair or nail clippings.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:
1. A composition comprising one or more of:

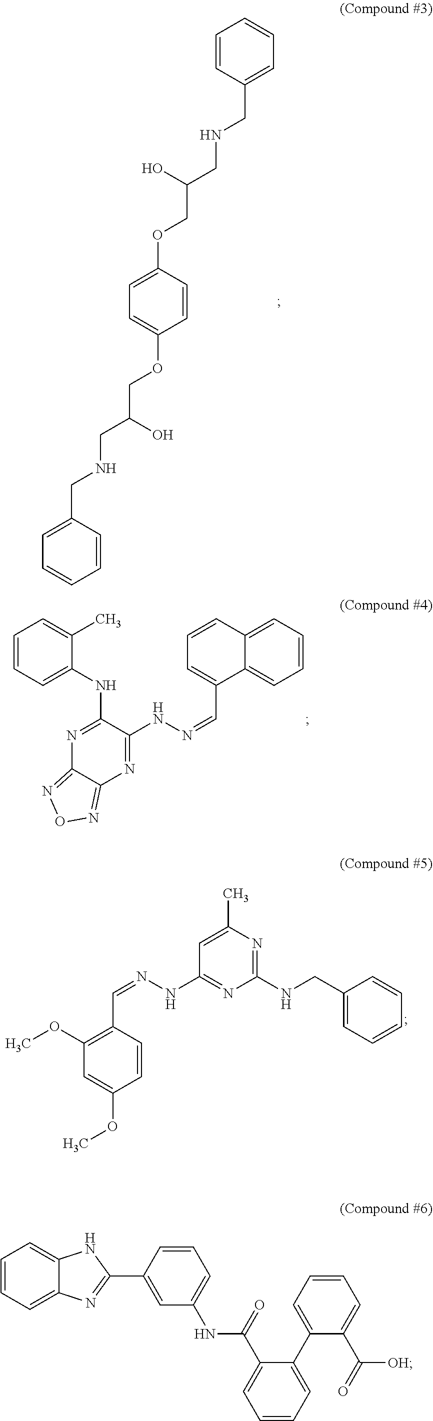

or a prodrug, isomer, enantiomer, diastereomer, racemate, tautomer, derivative, or salt thereof.
2. A composition or combination comprising:
   a) the composition of any one of the preceding claims; and
   b) one or more of:
      a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF).
3. A method of treating or preventing immune-related diseases, type 1 diabetes, transplant rejection, or lung fibrosis in a subject in need thereof, the method comprising administering a composition or combination of any one of the preceding paragraphs to the subject.
4. A method of slowing or delaying aging processes or extending lifespan in a subject, the method comprising administering a composition or combination of any one of the preceding paragraphs to the subject.
5. The method of any of paragraphs 3-4, wherein the subject has or has been determined to have rs3751143.
6. The method of any of paragraphs 3-5, wherein the subject has hyper-Th17 syndrome.
7. A method of treating or preventing immune-related diseases, type 1 diabetes, transplant rejection, or lung fibrosis in a subject in need thereof, the method comprising administering a composition comprising one or more of oATP, CE-224535, AZD9056, GSK1482160, a polypeptide comprising the ectodomain of P2X7R, and a P2X7R soluble protein to the subject;
   wherein the subject has or has been determined to have rs3751143.
8. A method of slowing or delaying aging processes or extending lifespan in a subject, the method comprising administering a composition comprising one or more of oATP, CE-224535, AZD9056, GSK1482160, a polypeptide comprising the ectodomain of P2X7R, and a P2X7R soluble protein to the subject;
   wherein the subject has or has been determined to have rs3751143.
9. The method of any of paragraphs 7-8, wherein the subject has Hyper-Th17 syndrome.
10. The method of any of paragraphs 3-9, wherein the treatment or prevention of type 1 diabetes comprises slowing the progression or onset of type 1 diabetes.
11. The method of any of paragraphs 3-9, wherein the subject has previously received a transplant of an organ and/or cells and the treatment or prevention of transplant rejection comprises suppressing or delaying rejection of the transplant.

In some embodiments, the present technology may be defined in any of the following numbered paragraphs:
1. A composition comprising one or more of:

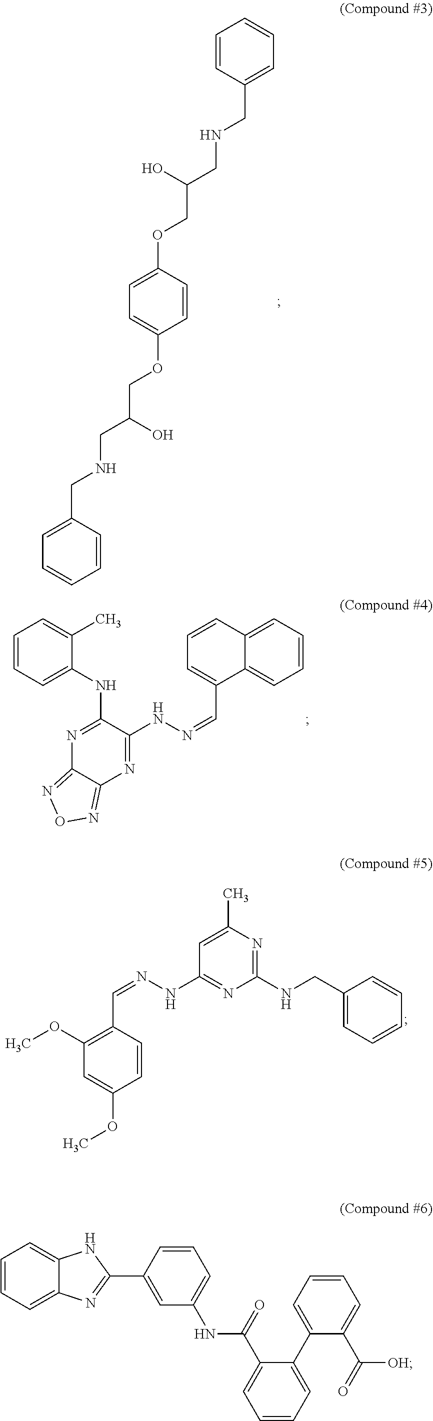

or a prodrug, isomer, enantiomer, diastereomer, racemate, tautomer, derivative, or salt thereof.
2. A composition or combination comprising:
   a) the composition of any one of the preceding paragraphs; and
   b) one or more of:
      a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF).
3. A method of treating or preventing immune-related diseases, type 1 diabetes, transplant rejection, or lung fibrosis in a subject in need thereof, the method comprising administering a composition or combination of any one of the preceding paragraphs to the subject.
4. A method of slowing or delaying aging processes or extending lifespan in a subject, the method comprising administering a composition or combination of any one of the preceding paragraphs to the subject.
5. The method of any of paragraphs 3-4, wherein the subject has or has been determined to have rs3751143.
6. The method of any of paragraphs 3-5, wherein the subject has hyper-Th17 syndrome.
7. A method of treating or preventing immune-related diseases, type 1 diabetes, transplant rejection, or lung fibrosis in a subject in need thereof, the method comprising administering a composition comprising one or more of oATP, CE-224535, AZD9056, GSK1482160, a polypeptide comprising the ectodomain of P2X7R, and a P2X7R soluble protein to the subject;
   wherein the subject has or has been determined to have rs3751143.
8. A method of slowing or delaying aging processes or extending lifespan in a subject, the method comprising administering a composition comprising one or more of oATP, CE-224535, AZD9056, GSK1482160, a polypeptide comprising the ectodomain of P2X7R, and a P2X7R soluble protein to the subject;
   wherein the subject has or has been determined to have rs3751143.
9. The method of any of paragraphs 7-8, wherein the subject has Hyper-Th17 syndrome.
10. The method of any of paragraphs 3-9, wherein the treatment or prevention of type 1 diabetes comprises slowing the progression or onset of type 1 diabetes.
11. The method of any of paragraphs 3-9, wherein the subject has previously received a transplant of an organ and/or cells and the treatment or prevention of transplant rejection comprises suppressing or delaying rejection of the transplant.
12. The composition or combination of any one of paragraphs 1-2, for use in the treatment or prevention of immune-related diseases, type 1 diabetes, transplant rejection, or lung fibrosis in a subject in need thereof.
13. The composition or combination of any one of paragraphs 1-2, for use in slowing or delaying aging processes or extending lifespan in a subject.
14. The composition or combination of any one of paragraphs 12-13, wherein the subject has or has been determined to have rs3751143.
15. The composition or combination of any one of paragraphs 12-14, wherein the subject has hyper-Th17 syndrome.
16. A composition comprising one or more of oATP, CE-224535, AZD9056, GSK1482160, a polypeptide comprising the ectodomain of P2X7R, and a P2X7R soluble protein, for use in treating or preventing immune-related diseases, type 1 diabetes, transplant rejection, or lung fibrosis in a subject determined to have rs3751143.
17. A composition comprising one or more of oATP, CE-224535, AZD9056, GSK1482160, a polypeptide comprising the ectodomain of P2X7R, and a P2X7R soluble protein, for use in slowing or delaying aging processes or extending lifespan in a subject determined to have rs3751143.
18. The composition of any one of paragraphs 16-17, wherein the subject has Hyper-Th17 syndrome.
19. The composition or combination of any one of paragraphs 12-18, wherein the treatment or prevention of type 1 diabetes comprises slowing the progression or onset of type 1 diabetes.

The technology described herein is further illustrated by the following examples which in no way should be construed as being further limiting.

### EXAMPLES

### Example 1

In addition to previously described purinergic inhibitors, described herein are extracellular ATP-binding small molecules as a new approach to treating immune-mediated diseases, like type 1 diabetes, transplant rejection, lung fibrosis and aging. It is further contemplated that such molecules can be used in combination with purinergic receptor inhibitors. The compositions and methods described herein are also contemplated for treating immunological abnormalities (Hyper-Th 17 syndrome) in subjects carrying the P2XTR mutation rs3751143.

Targeting eATP/P2X7R Axis with Small Molecules

Described herein is a method of treating type 1 diabetes, transplant rejection, lung fibrosis and aging in a subject, comprising administering a therapeutically effective dosage of an extracellular ATP-binding small molecule as described herein and/or a purinergic receptor inhibitor to the subject, wherein the purinergic receptor inhibitor is selected from the group consisting of: oATP; CE-224535; AZD9056, GSK1482160; a polypeptide comprising the ectodomain of P2X7R; and a P2X7R soluble protein.

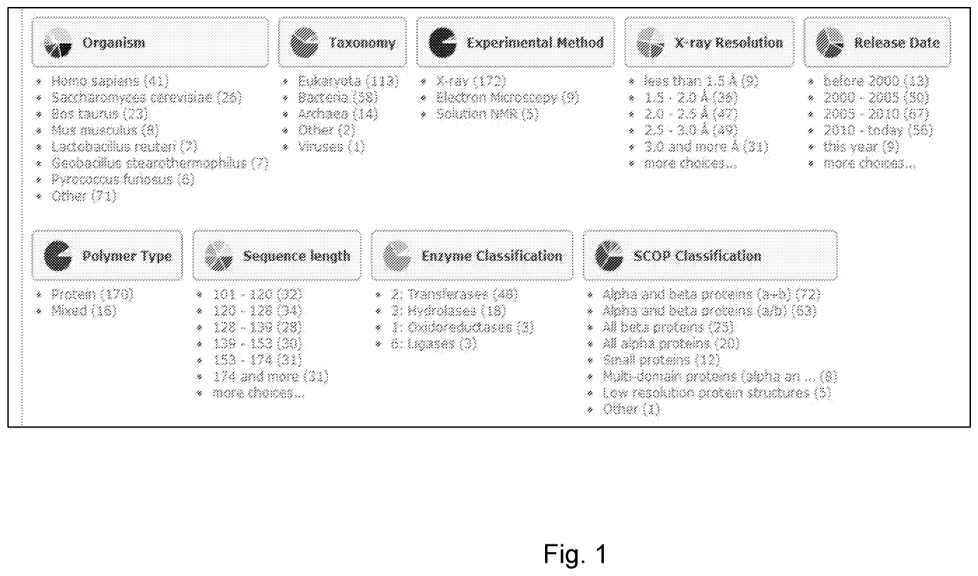

A small molecule library was screened using a bioinformatic approach, which demonstrated that numerous small molecules (size<200 residues) are capable of selectively binding the extracellular ATP (eATP) molecule, thereby preventing its binding to P2X7R and the activation of the downstream signaling (FIG. 1).

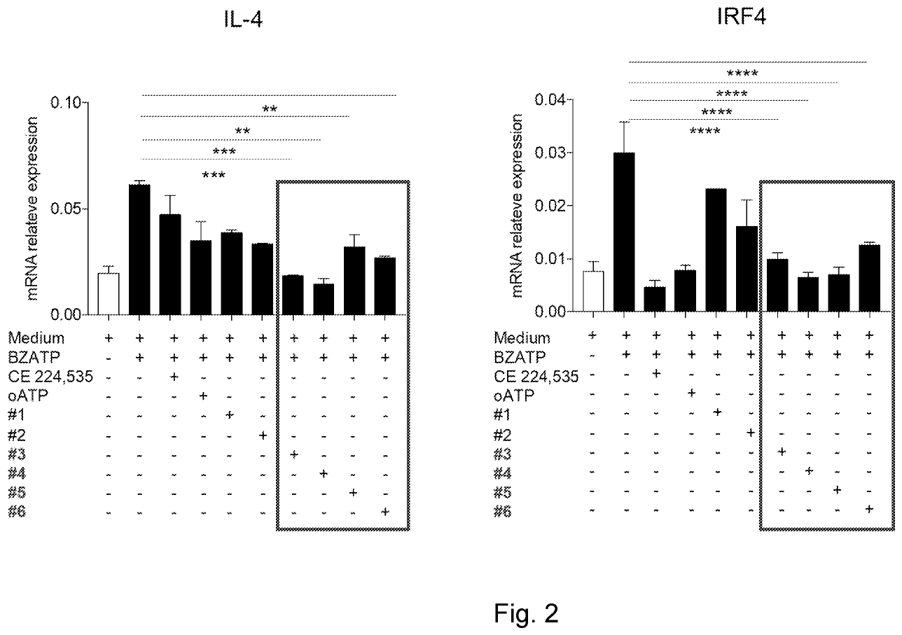

In order to specifically detect therapeutically effective molecules, High-Throughput Virtual Screening (HTVS) was performed. This led to the identification of 30 compounds with high binding affinity scores for the allosteric pocket of human P2X7R trimer (FIG. 2). This indicates that those small molecules could act as inhibitors of the eATP/P2X7R axis and thus could be included as P2X7R inhibitors to treat type 1 diabetes, transplant rejection, lung fibrosis and aging. Among these 30 most promising compounds, 6 with the highest affinity for the eATP/P2X7R axis, were tested in vitro.

Peripheral blood mononuclear cell (PBMCs) were isolated from collected human blood samples and recovered CD4+ T cells were purified and treated with different compounds. In particular cells were activated upon stimulation with benzoyl ATP (BzATP) and treated with human P2X7R inhibitor CE-224,535, P2X7 receptor antagonist oATP and the 6 small molecules identified in the bioinformatics and HTVS approaches above. The analysis of the expression of Th2-related factors IL-4 and IRF4 showed that compounds #3, #4, #5 and #6 have a significative ability to inhibit the activation of P2X7R signaling through eATP (FIG. 2). The chemical name and molecular formula of the Top 4 compounds are displayed in Table 1, while their structure is depicted above herein.

TABLE 1
Chemical name and molecular formula of compounds identified in screen

| | | Molecular |
|---|---|---|
| No. | CHEM NAME | Formula |
| #3 | 1-Benzylamino-3-[4-(3-benzylamino-2-hydroxy-propoxy)- | C26H32O4N2 |
| | phenoxy]-propan-2-ol | |
| #4 | [6-(N′-Naphthalen-1-ylmethylene-hydrazino)- | C22H17O1N7 |
| | [1,2,5]oxadiazolo[3,4-b]pyrazin-5-yl]-o-tolyl-amine | |
| #5 | Benzyl-{4-[N′-(2,4-dimethoxy-benzylidene)-hydrazino]-6- | C21H23O2N5 |
| | methyl-pyrimidin-2-yl}-amine | |
| #6 | 2′-[3-(1H-Benzoimidazol-2-yl)-phenylcarbamoyl]-biphenyl-2- | C27H19O3N3 |
| | carboxylic acid | |

Targeting eATP/P2X7R Axis to Treat Immunological Abnormalities (Hyper-Th17 Syndrome) in Subjects Carrying the P2X7R Mutation Rs3751143

Described herein is a method of treating immunological abnormalities (Hyper-Th17 syndrome) in a subject carrying the P2X7R mutation rs3751143 comprising administering a therapeutically effective dosage of an extracellular ATP-binding small molecule as described herein and/or a purinergic receptor inhibitor to the subject, wherein the purinergic receptor inhibitor is selected from the group consisting of: oATP; CE-224535; AZD9056, GSK1482160; a polypeptide comprising the ectodomain of P2X7R; and a P2X7R soluble protein.

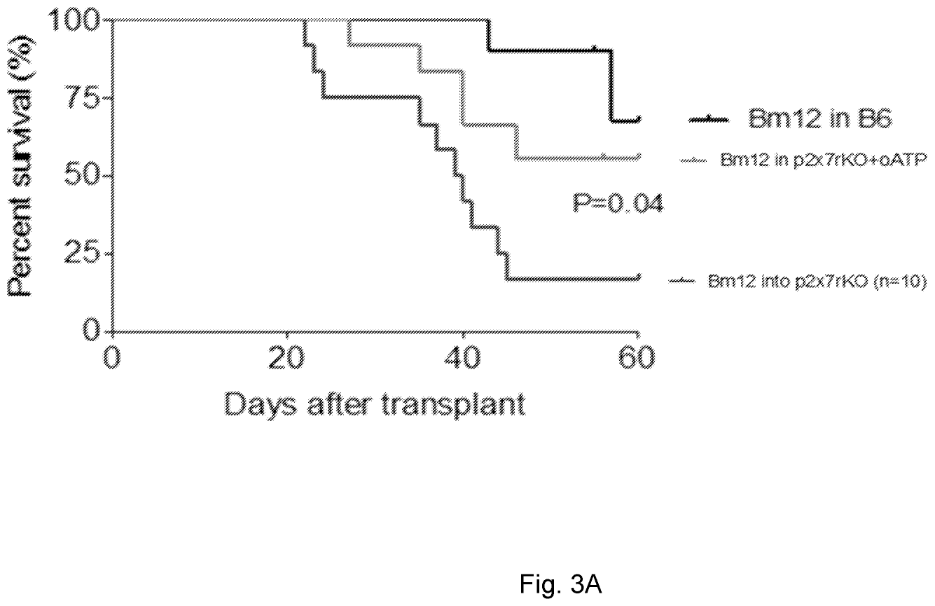

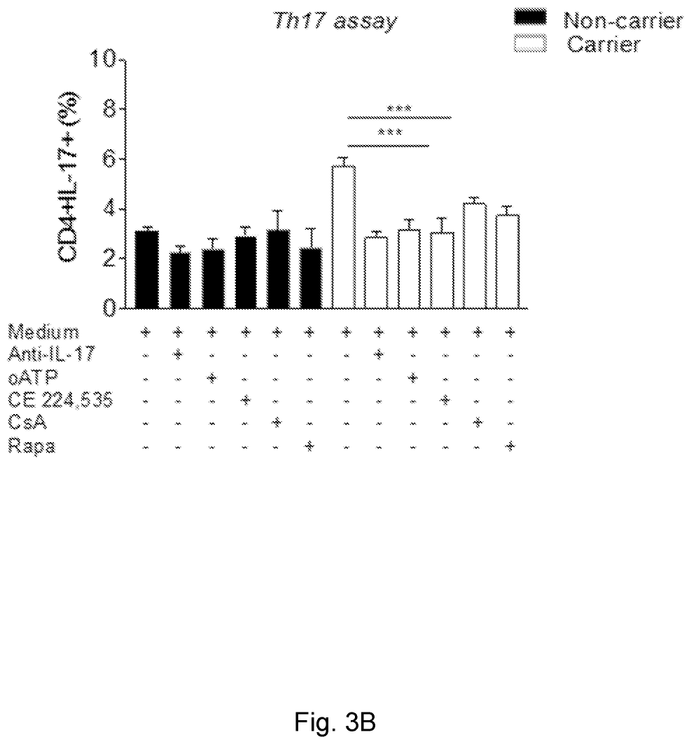

The P2X7R mutation rs3751143 is associated with the development of increased immunological events, particularly with the development of a HyperTh-17 syndrome, which is mainly characterized by an imbalance between T-helper cells which favors generation of Th-17 cells. Additionally, P2X7R inhibition with oATP or with CE 224,535 is capable of neutralizing activation of P2X7R signaling through eATP in cells of individuals who do not carry the P2X7R mutation¹⁰ (FIG. 3A). Interestingly, P2X7R^{-/-} mice receiving a heart transplantation from a Bm12 donor, which are prone to develop a Hyper-Th17 syndrome, benefit from treatment with the P2X7R inhibitor oATP. Specifically, such treatment significantly prolongs allograft survival (p=0.04, FIG. 3A). Moreover, it was demonstrated that the increased Th-17 generation observed in CD4⁺ T cells obtained from individuals carrying the P2X7R mutation is at least partially inhibited by adding a P2X7R inhibitor within the cell culture (oATP, CE 224535), thus emphasizing the benefits that P2X7R blockade has in preventing the development of Hyper-Th17 syndrome in individuals carrying the mutant allele (FIG. 3B).

Targeting eATP/P2X7R Axis to Increase Longevity and Prolong Survival

Described herein is a method of delaying cell metabolism thereby increasing longevity/prolonging survival in a subject comprising administering a therapeutically effective dosage of an extracellular ATP-binding small molecule as described herein and/or a purinergic receptor inhibitor to the subject, wherein the purinergic receptor inhibitor is selected from the group consisting of: oATP; CE-224535; AZD9056, GSK1482160; a polypeptide comprising the ectodomain of P2X7R; and a P2X7R soluble protein.

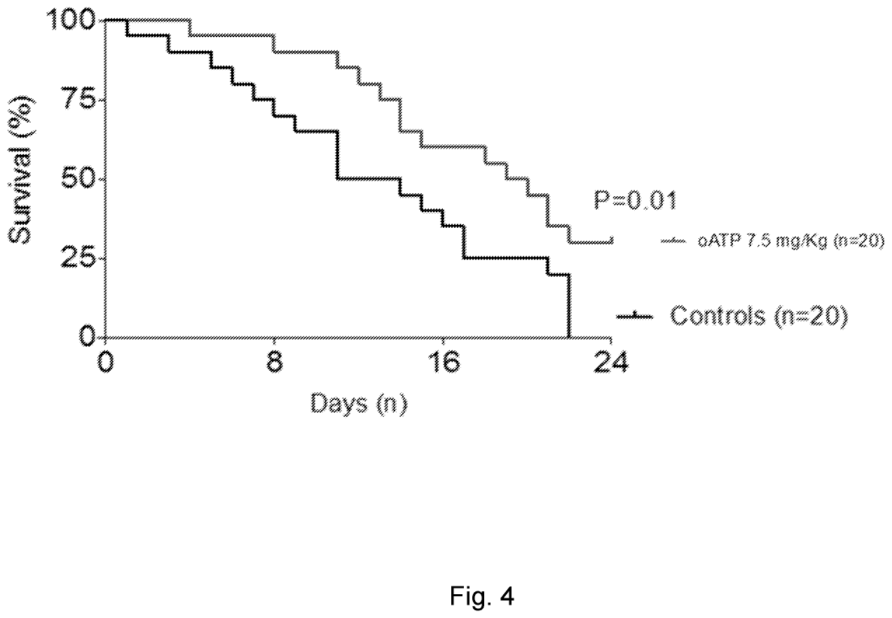

Administration of a P2X7R inhibitor (oATP) at a dose of 7.5 mg/Kg in n=20 C57BL6 (B6) mice significantly prolongs survival as compared to n=20 untreated controls (MST=12.5 vs. 19.5 weeks, p=0.01) as shown in FIG. 4. Interestingly, at 17 weeks 60% of treated animals were alive as compared to 25% in the untreated group, thus confirming a beneficial effect of P2X7R inhibition. Given that administration of P2X7R inhibitors is associated with a slow-down of metabolic processes within activated cells/tissues (9) and the effect of eATP in favoring cell senescence, it is contemplated herein that this mechanism could be responsible for promoting longevity and prolonging survival.

### Discussion

The results provided herein demonstrate that the eATP/P2X7R-axis is involved in immune-mediated diseases, in particular type 1 diabetes, transplant rejection, lung fibrosis and aging, thus targeting this signaling is a therapeutic option. Because of the highly targeted molecular effects and the favorable bioavailability with the potential of topical application, ATP-binding small molecules are particularly suitable for such therapies.

### REFERENCES

1. Eltzschig H K, Sitkovsky M V, Robson S C, N Engl J Med. 2012 Dec. 13; 367 (24): 2322-33
2. Elliott M R, Chekeni F B, Trampont P C, Nature 2009, 461:282-286
3. Piccini A, Carta S, Tassi S, Procl Natl Acad Sci USA 2008; 105:8067-8072
4. Junger W G, Nat Rev Immunol 2011; 11 (3): 201-212
5. Sullivan Ja, Am J Transplant 2014; 14 (7): 1512-1522
6. Atarashi K, Nature 2008; 455 (7214): 808-812
7. Vergani A, Tezza S, Fiorina P, Circulation 2013; 127 (4): 463-475
8. Sullivan J A, Adams A B, Transplantation 2014; 97 (5): 483-489
9. Tezza S, Ben Nasr M, Fiorina P, Diabetes 2018; 67 (10): 2038-2053
10. D'Addio F, Vergani A, Fiorina P, J Clin Invest 2018; 128 (8): 3490-3503

1. A composition comprising one or more of: 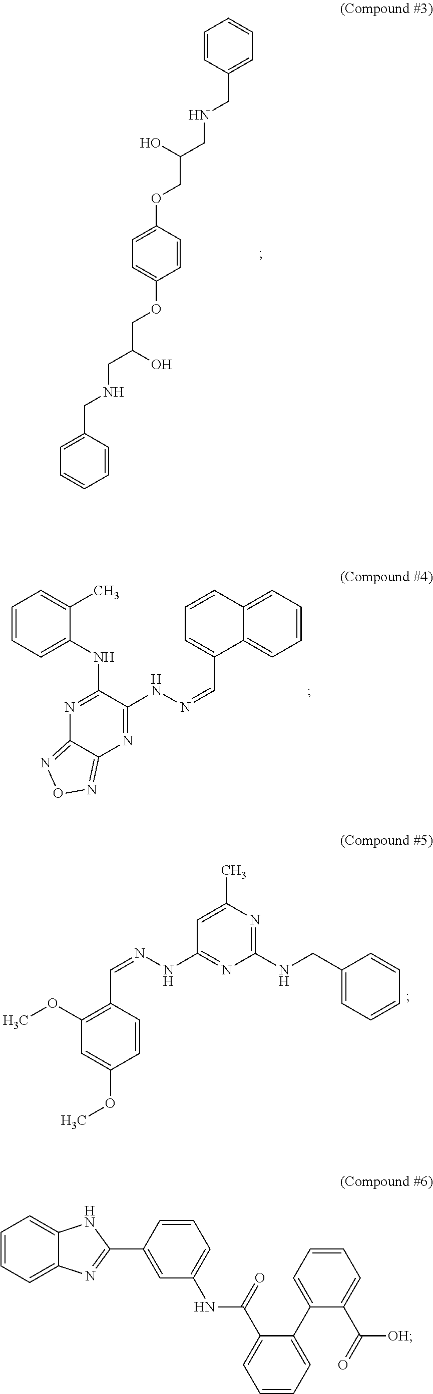 [SMILES: OC(CNCc1ccccc1)COc1ccc(OCC(O)CNCc2ccccc2)cc1 ; Cc1ccccc1Nc1nc2nonc2nc1N/N=C\c1cccc2ccccc12 ; COc1ccc(/C=N\Nc2cc(C)nc(NCc3ccccc3)n2)c(OC)c1 ; O=C(O)c1ccccc1-c1ccccc1C(=O)Nc1cccc(-c2nc3ccccc3[nH]2)c1]
or a prodrug, isomer, enantiomer, diastereomer, racemate, tautomer, derivative, or salt thereof.

2. A composition or combination comprising:
a) the composition of claim 1; and
b) one or more of:
a population of transplantation cells, rapamycin, oATP, CE-224535, AZD9056, GSK1482160, a polypeptide comprising the ectodomain of P2X7R, a P2X7R soluble protein, a steroid, teplizumab, rituximab, anti-thymocyte globulin (ATG), and mycophenolate mofetil (MMF).

3. A method of treating or preventing immune-related diseases, type 1 diabetes, transplant rejection, or lung fibrosis in a subject in need thereof, the method comprising administering a composition or combination of claim 1 to the subject.

4. A method of slowing or delaying aging processes or extending lifespan in a subject, the method comprising administering a composition or combination of claim 1 to the subject.

5. The method of claim 3, wherein the subject has or has been determined to have rs3751143.

6. The method of claim 3, wherein the subject has hyper-Th17 syndrome.

7. A method of treating or preventing immune-related diseases, type 1 diabetes, transplant rejection, or lung fibrosis in a subject in need thereof, the method comprising administering a composition comprising one or more of oATP, CE-224535, AZD9056, GSK1482160, a polypeptide comprising the ectodomain of P2X7R, and a P2X7R soluble protein to the subject;
wherein the subject has or has been determined to have rs3751143.

8. A method of slowing or delaying aging processes or extending lifespan in a subject, the method comprising administering a composition comprising one or more of oATP, CE-224535, AZD9056, GSK1482160, a polypeptide comprising the ectodomain of P2X7R, and a P2X7R soluble protein to the subject;
wherein the subject has or has been determined to have rs3751143.

9. The method of claim 7, wherein the subject has Hyper-Th17 syndrome.

10. The method of claim 3, wherein the treatment or prevention of type 1 diabetes comprises slowing the progression or onset of type 1 diabetes.

11. The method of claim 3, wherein the subject has previously received a transplant of an organ and/or cells and the treatment or prevention of transplant rejection comprises suppressing or delaying rejection of the transplant.

12.-19. (canceled)

20. The method of claim 4, wherein the subject has or has been determined to have rs3751143.
